(19) United States
(12) Patent Application Publication
(10) Pub. No.: US 2019/0385700 A1
(43) Pub. Date: Dec. 19, 2019

(54) METHODS AND SYSTEMS FOR DETERMINING The CELLULAR ORIGIN OF CELL-FREE NUCLEIC ACIDS

(71) Applicant: GUARDANT HEALTH, INC., Redwood City, CA (US)

(72) Inventors: Ariel JAIMOVICH, Redwood City, CA (US); Andrew KENNEDY, La Jolla, CA (US); Justin I. ODEGAARD, Redwood City, CA (US)

(21) Appl. No.: 16/429,997

(22) Filed: Jun. 3, 2019

Related U.S. Application Data
(60) Provisional application No. 62/680,301, filed on Jun. 4, 2018.

(51) Int. Cl. G16B 30/10; C12Q 1/6809; G16B 40/00; G16B 50/00; G16H 10/60; G16H 50/20

(52) U.S. Cl. CPC G16B 30/10; C12Q 1/6809; G16H 50/20; G16B 50/00; G16H 10/60; G16B 40/00

(57) ABSTRACT

Provided herein are methods that are useful in determining the cellular origin of cell-free nucleic acid (cfNA) fragments from cfNA samples, such as liquid biopsy samples. The methods disclosed herein typically improve the specificity and/or sensitivity of assays for detecting diseased cell nucleic acids (e.g., cancer cell DNA) in cfNA samples by identifying variant alleles produced by non-target cells, such as hematopoietic stem cells, in certain embodiments. Yet other aspects include related systems and computer readable media, among numerous other applications.

### BACKGROUND

The detection and quantification of polynucleotides is important for molecular biology and medical applications, such as diagnostics. Genetic testing is particularly useful for a number of diagnostic methods. For example, disorders that are caused by rare genetic alterations (e.g., sequence variants) or changes in epigenetic markers, such as cancer and partial or complete aneuploidy, may be detected or more accurately characterized with DNA sequence information.

Early detection and monitoring of genetic diseases, such as cancer, is often needed in the successful treatment or management of the disease. One approach may include the monitoring of a sample derived from cell-free nucleic acids, a population of polynucleotides that can be found in different types of bodily fluids. In some cases, disease may be characterized or detected based on detection of genetic aberrations, such as copy number variation and/or sequence variation of one or more nucleic acid sequences, or the development of other genetic alterations. Cell-free DNA (cfDNA) may contain genetic aberrations associated with a particular disease.

CfDNA present in blood, however, can originate from several cell sources, both cancerous and noncancerous cells. One source of cell free DNA that can be problematic is hematopoietic stem cells, mutations in which might lead to the expansion of a clonal population of blood cells. Such acquisition of somatic mutations that drive clonal expansion, without other signs of hematologic malignancies, is referred to as cells that result from “Clonal Hematopoiesis of Indeterminate Potential” (CHIP). See, Steensma et al, Blood, 126:9-16 (2015). At least 10% of the elderly population above the age of 70 carry CHIP due to oligoclonal expansion of mutated hematopoietic stem cells. See, Jaiswal et al., N. Engl. J. Med., 371(26):2488-2498 (2014). Hematopoietic stem cells can contain genetic variants in regions of the genome associated with cancer, even though the hematopoietic stem cells are not cancerous. Accordingly, it is of interest to identify alleles that are predominantly present in hematopoietic stem cells, but absent in cancer cells that contribute to sampled cfDNA populations.

### SUMMARY

The present disclosure provides methods, computer readable media, and systems that are useful in determining the cellular origin of cell-free nucleic acid (cfNA) fragments from cfNA samples, such as liquid biopsy samples. These aspects typically improve the specificity and/or sensitivity of assays for detecting diseased cell nucleic acids (e.g., cancer cell DNA) in cfNA samples by identifying variant alleles produced by non-target cells, such as hematopoietic stem cells, in certain embodiments. Further, the methods disclosed herein facilitate the identification of the cellular source of nucleic acids, which are often present in very small quantities in cfNA samples, such as in the case of tumor originating nucleic acids from early stage cancers. Accordingly, the methods and related aspects disclosed herein foster the early detection of disease, among numerous other applications.

In one aspect, this disclosure provides a method of detecting a nucleic acid molecule that originates from a target cell in a subject at least partially using a computer. The method includes (a) receiving, by the computer, test sequence information comprising sequence reads obtained from cell-free nucleic acid (cfNA) fragments from a test sample obtained from the subject. The method also includes (b) identifying a presence of at least one allelic variant in the test sequence information that substantially matches at least one classification allele on a target nucleic acid variant filter list. The classification allele comprises a subclonality score below at least one selected cutoff threshold value thereby indicating that the classification allele is from a reference cfNA fragment that originates from the target cell, thereby detecting the nucleic acid molecule that originates from the target cell in the subject. In some embodiments, for example, (b) includes identifying at least one allelic variant in the test sequence information; mapping the allelic variant to at least one classification allele on a target nucleic acid variant filter list; identifying a subclonality score of the classification allele; and comparing the subclonality score to at least one selected cutoff threshold value, wherein when the subclonality score is below the selected cutoff threshold value it indicates that the classification allele is from a reference cfNA fragment that originates from the target cell.

In one aspect, this disclosure provides a method of detecting a nucleic acid molecule that originates from a tumor cell in a subject at least partially using a computer. The method includes (a) receiving, by the computer, test sequence information comprising sequence reads obtained from cell-free deoxyribonucleic acid (cfDNA) fragments in a test sample obtained from the subject. The methods also includes (b) removing (e.g., deleting, suppressing, ignoring, or the like), by the computer, one or more of the sequence reads (e.g., that comprise at least portions of classification alleles) that originate from a hematopoietic stem cell of the subject from the test sequence information to generate filtered test sequence information. In addition; the method also includes (c) identifying, by the computer, a presence of one or more of the sequence reads in the filtered test sequence information that substantially align with reference sequence information obtained from one or more reference subjects, which reference sequence information originates from one or more tumor cells in the reference subjects, thereby detecting the nucleic acid molecule that originates from the tumor cell in the subject.

In one aspect, this disclosure provides a method of treating a disease in a subject. The method includes (a) receiving test sequence information comprising sequence reads obtained from cell-free nucleic acid (cfNA) fragments from a test sample obtained from the subject. The method also includes (b) identifying a presence of at least one allelic variant in the test sequence information that substantially matches at least one classification allele on a target nucleic acid variant filter list. The classification allele comprises a subclonality score below at least one selected cutoff threshold value thereby indicating that the classification allele is from a reference cfNA fragment that originates from a diseased cell, thereby diagnosing the disease in the subject. In addition, the method also includes (c) administering one or more therapies to the subject, thereby treating the disease in the subject.

In another aspect, the disclosure provides a method of generating a classifier, or at least a portion thereof, at least partially using a computer. The method includes (a) generating, by the computer, a subclonality score for each allele in a set of classification alleles from sequence information comprising sequence reads obtained from cell-free nucleic acid (cfNA) fragments from one or more reference samples, wherein each classification allele is of potential clinical significance and comprises a minor allele observed at a given locus in the reference samples. The method also includes (b) comparing, by the computer, at least one selected cutoff threshold value to the subclonality scores, wherein classification alleles with subclonality scores above the selected cutoff threshold value indicate that those classification alleles are from reference cfNA fragments originating from non-target cells, which classification alleles are added to a non-target nucleic acid variant filter list, and/or wherein classification alleles with subclonality scores below the cutoff threshold value indicate that those classification alleles are from reference cfNA fragments originating from target cells, which classification alleles are added to a target nucleic acid variant filter list, thereby generating the classifier.

In another aspect, the disclosure provides a method of generating a classifier, or at least a portion thereof, at least partially using a computer. The method includes (a) identifying, by the computer, a set of classification alleles from sequence information comprising sequence reads obtained from cell-free nucleic acid (cfNA) fragments from one or more reference samples, wherein each classification allele is of potential clinical significance and comprises a minor allele observed at a given locus in the reference samples. The method also includes (b) determining, by the computer, a value of a minor allele frequency (MAF) for each classification allele in each of the reference samples from the sequence information, and (c) determining, by the computer, a value of a maximum minor allele frequency (maxMAF) for each of the reference samples. The method also includes (d) calculating, by the computer, for each classification allele observed in a given reference sample, a ratio of the value of the MAF over the value of the maxMAF for at least a portion of the reference samples to generate ratio values. The method also includes (e) calculating, by the computer, for each of the classification alleles, a ratio of a number of times a given classification allele in at least the portion of the reference samples had a ratio value below at least one selected clonality border value over a total number of times the given classification allele occurred in at least the portion of the reference samples to generate a subclonality score for each of the classification alleles in at least the portion of the reference samples. In addition, the method also includes (f) comparing, by the computer, at least one selected cutoff threshold value to the subclonality scores, wherein classification alleles with subclonality scores above the selected cutoff threshold value indicate that those classification alleles are from reference cfNA fragments originating from non-target cells, which classification alleles are added to a non-target nucleic acid variant filter list, and/or wherein classification alleles with subclonality scores below the cutoff threshold value indicate that those classification alleles are from reference cfNA fragments originating from target cells, which classification alleles are added to a target nucleic acid variant filter list, thereby generating the classifier.

In another aspect, the disclosure provides a method of producing a database of subclonality scores of use in classifying a cellular origin of cell-free nucleic acid (cfNA) fragments in test samples obtained from subjects. The method includes (a) identifying, by a computer, a set of classification alleles from sequence information comprising sequence reads obtained from cell-free nucleic acid (cfNA) fragments from one or more reference samples, wherein each classification allele is of potential clinical significance and comprises a minor allele observed at a given locus in the reference samples. The method also includes (b) determining, by the computer, a value of a minor allele frequency (MAF) for each classification allele in each of the reference samples from the sequence information, c) determining, by the computer, a value of a maximum minor allele frequency (maxMAF) for each of the reference samples, and (d) calculating, by the computer, for each classification allele observed in a given reference sample, a ratio of the value of the MAF over the value of the maxMAF for at least a portion of the reference samples to generate ratio values. The method also includes (e) calculating, by the computer, for each of the classification alleles, a ratio of a number of times a given classification allele in at least the portion of the reference samples had a ratio value below at least one selected clonality border value over a total number of times the given classification allele occurred in at least the portion of the reference samples to generate a subclonality score for each of the classification alleles in at least the portion of the reference samples. In addition, the method also includes (f) storing, non-transiently, the subclonality scores indexed to corresponding classification alleles in a database system, thereby producing the database of subclonality scores to use in classifying the cellular origin of cfNA fragments in test samples obtained from subjects.

In some embodiments, the methods disclosed herein include identifying the set of classification alleles comprises determining a value of an MAF for each somatic nucleic acid variant at each locus in a set of target genomic loci of potential clinical significance from the sequence information obtained from the reference samples, wherein the set of target genomic loci is identical in each reference sample, and determining a value of a maxMAF for each of the reference samples, to generate allelic information. In certain embodiments, the MAF for each classification allele is less than about 2%. In some embodiments, the MAF for each classification allele is less than about 1%.

In certain embodiments, the methods disclosed herein include using clinical information indexed to the reference samples to generate the classifier. In some embodiments, the methods disclosed herein include using clinical information indexed to the test sample to detect the nucleic acid molecule that originates from the target cell in the subject. In certain embodiments, the clinical information is selected from the group consisting of: age, gender, race, weight, body mass index (BMI), clinical history, tobacco usage, alcohol usage, and the like. In other exemplary embodiments, subclonal lists (e.g., target or non-target nucleic acid variant filter lists) are generated over different subsets of samples based on, for example, minimal maxMAF, calling maxMAF based on known driver mutations, and/or the like. In some embodiments, subclonal lists are generated based specific indications, such as a given cancer-type (e.g., lung, colorectal, etc.). In certain embodiments, machine learning classifiers are trained based upon one or more features, including mutant allele frequency, subclonal ratio, gene type, variants associated with hematological malignancies, patient age, observation of other CHIP variants, cancer type, and/or the like.

In some embodiments, the methods disclosed herein include determining subclonality scores using frequencies of each MAF/max-MAF value for each of the classification alleles. In certain embodiments, the selected clonality border value is in a range of about 1% to about 99%. In some of these embodiments, for example, the selected clonality border value is about 10%, about 20%, about 30%, about 40%, about 50%, about 60%, about 70%, about 80%, or about 90%. In some embodiments, the selected cutoff threshold value is in a range of about 1% to about 99%. In some of these embodiments, for example, the selected cutoff threshold value is about 10%, about 20%, about 30%, about 40%, about 50%, about 60%, about 70%, about 80%, or about 90%.

In certain embodiments, the methods disclosed herein include comparing the subclonality scores to multiple selected cutoff threshold values. In some of these embodiments, for example, the multiple selected cutoff threshold values comprise a first cutoff threshold value and a second cutoff threshold value, which first cutoff threshold value is greater than the second cutoff threshold value, wherein classification alleles with subclonality scores above the first cutoff threshold value indicate that those classification alleles are from reference cfNA fragments originating from non-target cells, which classification alleles are added to the non-target nucleic acid variant filter list, and/or wherein classification alleles with subclonality scores below the second cutoff threshold value indicate that those classification alleles are from reference cfNA fragments originating from target cells which classification alleles are added to the target nucleic acid variant filter list.

In some embodiments, the methods disclosed herein include classifying an allelic variant in the test sequence information that substantially matches at least one classification allele on a non-target nucleic acid variant filter list as originating from a target cell when the allelic variant comprises an MAF greater than about 1%. In certain embodiments, the methods disclosed herein include classifying an allelic variant in the test sequence information that substantially matches at least one classification allele on a non-target nucleic acid variant filter list as originating from a target cell when the allelic variant comprises a truncation, an indel, and/or a splice site variant.

In certain embodiments, the methods disclosed herein include determining a frequency of each ratio value for a given classification allele in at least the portion of the reference samples. In some embodiments, the methods disclosed herein include using the classifier to determine whether a test sample obtained from a subject comprises cfNA fragments that originate from the target cells. In certain embodiments, the methods disclosed herein include using the classifier to determine whether a test sample obtained from a subject comprises cfNA fragments that originate from the non-target cells. In some embodiments, a database comprises the target nucleic acid variant filter list and/or the non-target nucleic acid variant filter list.

In certain embodiments, the non-target cells comprise non-diseased cells. In some embodiments, the non-target cells comprise hematopoietic stem cells. In certain embodiments, the non-target cells comprise non-tumor cells. In some embodiments, the non-target cells comprise maternal cells. In certain embodiments, the non-target cells comprise transplant recipient cells.

In certain embodiments, the target cells comprise diseased cells. In some embodiments, the target cells comprise tumor cells. In some embodiments, the target cells comprise fetal cells. In certain embodiments, the target cells comprise transplant donor cells.

In certain embodiments, the methods disclosed herein include treating diseases. In some of these embodiments, for example, the disease comprises cancer and wherein the therapies comprise at least one immunotherapy. Typically, the subject is a mammalian subject (e.g., a human subject).

In some embodiments, the methods disclosed herein further comprise obtaining the test sample from the subject. The test sample is typically selected from the group consisting of: blood, plasma, serum, sputum, urine, semen, vaginal fluid, feces, synovial fluid, spinal fluid, saliva, and the like. In some embodiments, the methods disclosed herein further comprise generating the test sequence information from the cfNA fragments in the test sample. In some embodiments, the methods disclosed herein further comprise amplifying segments of the cfNA fragments that comprise target genomic loci to generate amplified nucleic acids. In certain embodiments, the methods disclosed herein further comprise sequencing the cfNA fragments in the test sample to generate the test sequence information. In some of these embodiments, the test sequence information is obtained from targeted segments of the cfNA fragments in the test sample, wherein the targeted segments are obtained by selectively enriching one or more regions from the cfNA fragments in the test sample prior to sequencing. In certain embodiments, the methods disclosed herein further comprise amplifying the obtained targeted segments prior to sequencing. In certain embodiments, the methods disclosed herein further comprise attaching one or more adapters comprising barcodes to the cfNA fragments and/or the amplified targeted segments prior to sequencing. In certain embodiments, the sequencing is selected from the group consisting of: targeted sequencing, bisulfite sequencing, intron sequencing, exome sequencing, and whole genome sequencing.

In still another aspect, the disclosure provides a system that includes a controller comprising, or capable of accessing, computer readable media comprising non-transitory computer-executable instructions which, when executed by at least one electronic processor perform at least: (a) receiving test sequence information comprising sequence reads obtained from cell-free nucleic acid (cfNA) fragments from a test sample obtained from a subject, and (b) identifying a presence of at least one allelic variant in the test sequence information that substantially matches at least one classification allele on a target nucleic acid variant filter list, which classification allele comprises a subclonality score below at least one selected cutoff threshold value thereby indicating that the classification allele is from a reference cfNA fragment that originates from a target cell, thereby indicating that the allelic variant in the test sequence information originates from the target cell in the subject. In some embodiments, for example, (b) includes identifying at least one allelic variant in the test sequence information; mapping the allelic variant to at least one classification allele on a target nucleic acid variant filter list; identifying a subclonality score of the classification allele; and comparing the subclonality score to at least one selected cutoff threshold value, wherein when the subclonality score is below the selected cutoff threshold value it indicates that the classification allele is from a reference cfNA fragment that originates from the target cell.

In still another aspect, the disclosure provides a system that includes a controller comprising, or capable of accessing, computer readable media comprising non-transitory computer-executable instructions which, when executed by at least one electronic processor perform at least: (a) receiving test sequence information comprising sequence reads obtained from cell-free deoxyribonucleic acid (cfDNA) fragments in a test sample obtained from the subject, (b) removing (e.g., deleting, suppressing, ignoring, or the like) one or more of the sequence reads (e.g., that comprise at least portions of classification alleles) that originate from a hematopoietic stem cell of the subject from the test sequence information to generate filtered test sequence information, and (c) identifying a presence of one or more of the sequence reads in the filtered test sequence information that substantially align with reference sequence information obtained from one or more reference subjects, which reference sequence information originates from a tumor cell in the reference subjects, thereby indicating that the test sample comprises one or more cfDNA fragments that originate from the tumor cell in the subject.

In still another aspect, the disclosure provides a system that includes a controller comprising, or capable of accessing, computer readable media comprising non-transitory computer-executable instructions which, when executed by at least one electronic processor perform at least: (a) generating a subclonality score for each allele in a set of classification alleles from sequence information comprising sequence reads obtained from cell-free nucleic acid (cfNA) fragments from one or more reference samples, wherein each classification allele is of potential clinical significance and comprises a minor allele observed at a given locus in the reference samples, and (b) comparing at least one selected cutoff threshold value to the subclonality scores, wherein classification alleles with subclonality scores above the selected cutoff threshold value indicate that those classification alleles are from reference cfNA fragments originating from non-target cells, which classification alleles are added to a non-target nucleic acid variant filter list, and/or wherein classification alleles with subclonality scores below the cutoff threshold value indicate that those classification alleles are from reference cfNA fragments originating from target cells, which classification alleles are added to a target nucleic acid variant filter list.

In still another aspect, the disclosure provides a system that includes a controller comprising, or capable of accessing, computer readable media comprising non-transitory computer-executable instructions which, when executed by at least one electronic processor perform at least: (a) identifying a set of classification alleles from sequence information comprising sequence reads obtained from cell-free nucleic acid (cfNA) fragments from one or more reference samples, wherein each classification allele is of potential clinical significance and comprises a minor allele observed at a given locus in the reference samples, (b) determining a value of a minor allele frequency (MAF) for each classification allele in each of the reference samples from the sequence information, (c) determining a value of a maximum minor allele frequency (maxMAF) for each of the reference samples, (d) calculating for each classification allele observed in a given reference sample, a ratio of the value of the MAF over the value of the maxMAF for at least a portion of the reference samples to generate ratio values, (e) calculating, for each of the classification alleles, a ratio of a number of times a given classification allele in at least the portion of the reference samples had a ratio value below at least one selected clonality border value over a total number of times the given classification allele occurred in at least the portion of the reference samples to generate a subclonality score for each of the classification alleles in at least the portion of the reference samples, and, (f) comparing at least one selected cutoff threshold value to the subclonality scores, wherein classification alleles with subclonality scores above the selected cutoff threshold value indicate that those classification alleles are from reference cfNA fragments originating from non-target cells, which classification alleles are added to a non-target nucleic acid variant filter list, and/or wherein classification alleles with subclonality scores below the cutoff threshold value indicate that those classification alleles are from reference cfNA fragments originating from target cells, which classification alleles are added to a target nucleic acid variant filter list.

In some embodiments, the systems disclosed herein include a nucleic acid sequencer operably connected to the controller, which nucleic acid sequencer is configured to provide the sequence information from the cfNA fragments in the test sample and/or the reference samples. In certain of these embodiments, the nucleic acid sequencer is configured to perform pyrosequencing, bisulfite sequencing, single-molecule sequencing, nanopore sequencing, semiconductor sequencing, sequencing-by-ligation or sequencing-by-hybridization on the nucleic acids to generate sequencing reads.

In some embodiments, the systems disclosed herein include a sample preparation component operably connected to the controller, which sample preparation component is configured to prepare the cfNA fragments to be sequenced by a nucleic acid sequencer. In some of these embodiments, the sample preparation component is configured to selectively enrich regions from the cfNA fragments in the test sample and/or the reference samples. In certain embodiments, the sample preparation component is configured to attach one or adapters comprising barcodes to the cfNA fragments.

In certain embodiments, the systems disclosed herein include a nucleic acid amplification component operably connected to the controller, which nucleic acid amplification component is configured to amplify the cfNA fragments in the test sample and/or the reference samples. In some of these embodiments, the nucleic acid amplification component is configured to amplify selectively enriched regions from the cfNA fragments in the test sample and/or the reference samples. In some embodiments, the systems disclosed herein include a material transfer component operably connected to the controller, which material transfer component is configured to transfer one or more materials between a nucleic acid sequencer, a nucleic acid amplification component, and/or a sample preparation component. In certain embodiments, the systems disclosed herein include a database operably connected to the controller, which database comprises the non-target nucleic acid variant filter list, and/or the target nucleic acid variant filter list.

In still another aspect, the disclosure provides a computer readable media comprising non-transitory computer-executable instructions which, when executed by at least one electronic processor perform at least: (a) receiving test sequence information comprising sequence reads obtained from cell-free nucleic acid (cfNA) fragments from a test sample obtained from a subject, and (b) identifying a presence of at least one allelic variant in the test sequence information that substantially matches at least one classification allele on a target nucleic acid variant filter list, which classification allele comprises a subclonality score below at least one selected cutoff threshold value thereby indicating that the classification allele is from a reference cfNA fragment that originates from a target cell, thereby indicating that the allelic variant in the test sequence information originates from the target cell in the subject. In some embodiments, for example, (b) includes identifying at least one allelic variant in the test sequence information; mapping the allelic variant to at least one classification allele on a target nucleic acid variant filter list; identifying a subclonality score of the classification allele; and comparing the subclonality score to at least one selected cutoff threshold value, wherein when the subclonality score is below the selected cutoff threshold value it indicates that the classification allele is from a reference cfNA fragment that originates from the target cell.

In another aspect, the disclosure provides a computer readable media comprising non-transitory computer-executable instructions which, when executed by at least one electronic processor perform at least: (a) receiving test sequence information comprising sequence reads obtained from cell-free deoxyribonucleic acid (cfDNA) fragments in a test sample obtained from the subject, (b) removing (e.g., deleting, suppressing, ignoring, or the like) one or more of the sequence reads (e.g., that comprise at least portions of classification alleles) that originate from a hematopoietic stem cell of the subject from the test sequence information to generate filtered test sequence information, and (c) identifying a presence of one or more of the sequence reads in the filtered test sequence information that substantially align with reference sequence information obtained from one or more reference subjects, which reference sequence information originates from a tumor cell in the reference subjects, thereby indicating that the test sample comprises one or more cfDNA fragments that originate from the tumor cell in the subject.

In another aspect, the disclosure provides a computer readable media comprising non-transitory computer-executable instructions which, when executed by at least one electronic processor perform at least: (a) generating a subclonality score for each allele in a set of classification alleles from sequence information comprising sequence reads obtained from cell-free nucleic acid (cfNA) fragments from one or more reference samples, wherein each classification allele is of potential clinical significance and comprises a minor allele observed at a given locus in the reference samples, and (b) comparing at least one selected cutoff threshold value to the subclonality scores, wherein classification alleles with subclonality scores above the selected cutoff threshold value indicate that those classification alleles are from reference cfNA fragments originating from non-target cells, which classification alleles are added to a non-target nucleic acid variant filter list, and/or wherein classification alleles with subclonality scores below the cutoff threshold value indicate that those classification alleles are from reference cfNA fragments originating from target cells, which classification alleles are added to a target nucleic acid variant filter list.

In another aspect, the disclosure provides a computer readable media comprising non-transitory computer-executable instructions which, when executed by at least one electronic processor perform at least: (a) identifying a set of classification alleles from sequence information comprising sequence reads obtained from cell-free nucleic acid (cfNA) fragments from one or more reference samples, wherein each classification allele is of potential clinical significance and comprises a minor allele observed at a given locus in the reference samples, (b) determining a value of a minor allele frequency (MAF) for each classification allele in each of the reference samples from the sequence information, (c) determining a value of a maximum minor allele frequency (maxMAF) for each of the reference samples, (d) calculating for each classification allele observed in a given reference sample, a ratio of the value of the MAF over the value of the maxMAF for at least a portion of the reference samples to generate ratio values, (e) calculating, for each of the classification alleles, a ratio of a number of times a given classification allele in at least the portion of the reference samples had a ratio value below at least one selected clonality border value over a total number of times the given classification allele occurred in at least the portion of the reference samples to generate a subclonality score for each of the classification alleles in at least the portion of the reference samples, and (f) comparing at least one selected cutoff threshold value to the subclonality scores, wherein classification alleles with subclonality scores above the selected cutoff threshold value indicate that those classification alleles are from reference cfNA fragments originating from non-target cells, which classification alleles are added to a non-target nucleic acid variant filter list, and/or wherein classification alleles with subclonality scores below the cutoff threshold value indicate that those classification alleles are from reference cfNA fragments originating from target cells, which classification alleles are added to a target nucleic acid variant filter list.

In some embodiments of the system or computer readable media disclosed herein, the computer readable media include non-transitory computer-executable instructions which, when executed by the at least one electronic processor further perform at least: determining a value of an MAF for each somatic nucleic acid variant at each locus in a set of target genomic loci of potential clinical significance from the sequence information obtained from the reference samples, wherein the set of target genomic loci is identical in each reference sample, and determining a value of a maxMAF for each of the reference samples, to generate allelic information.

In certain embodiments of the system or computer readable media disclosed herein, the computer readable media include non-transitory computer-executable instructions which, when executed by the at least one electronic processor further perform at least: using clinical information indexed to the reference samples to generate the non-target nucleic acid variant filter list, and/or the target nucleic acid variant filter list. In certain embodiments of the system or computer readable media disclosed herein, the computer readable media include non-transitory computer-executable instructions which, when executed by the at least one electronic processor further perform at least: using clinical information indexed to the test sample to detect cfNA fragments that originate from the target cell in the subject. In certain embodiments of the system or computer readable media disclosed herein, the computer readable media include non-transitory computer-executable instructions which, when executed by the at least one electronic processor further perform at least: determining subclonality scores using frequencies of each MAF/max-MAF value for each of the classification alleles.

In certain embodiments of the system or computer readable media disclosed herein, the computer readable media include non-transitory computer-executable instructions which, when executed by the at least one electronic processor further perform at least: comparing the subclonality scores to multiple selected cutoff threshold values, wherein the multiple selected cutoff threshold values comprise a first cutoff threshold value and a second cutoff threshold value, which first cutoff threshold value is greater than the second cutoff threshold value, wherein classification alleles with subclonality scores above the first cutoff threshold value indicate that those classification alleles are from reference cfNA fragments originating from non-target cells, which classification alleles are added to the non-target nucleic acid variant filter list, and/or wherein classification alleles with subclonality scores below the second cutoff threshold value indicate that those classification alleles are from reference cfNA fragments originating from target cells which classification alleles are added to the target nucleic acid variant filter list. In certain embodiments of the system or computer readable media disclosed herein, the computer readable media include non-transitory computer-executable instructions which, when executed by the at least one electronic processor further perform at least: classifying an allelic variant in the test sequence information that substantially matches at least one classification allele on a non-target nucleic acid variant filter list as originating from a target cell when the allelic variant comprises an MAF greater than about 1%. In some embodiments of the system or computer readable media disclosed herein, the computer readable media include non-transitory computer-executable instructions which, when executed by the at least one electronic processor further perform at least: classifying an allelic variant in the test sequence information that substantially matches at least one classification allele on a non-target nucleic acid variant filter list as originating from a target cell when the allelic variant comprises a truncation, an indel, and/or a splice site variant.

In some embodiments of the system or computer readable media disclosed herein, the computer readable media include non-transitory computer-executable instructions which, when executed by the at least one electronic processor further perform at least: determining a frequency of each ratio value for a given classification allele in at least the portion of the reference samples. In certain embodiments of the system or computer readable media disclosed herein, the computer readable media include non-transitory computer-executable instructions which, when executed by the at least one electronic processor further perform at least: using the target nucleic acid variant filter list to determine whether a test sample obtained from a subject comprises cfNA fragments that originate from the target cells. In some embodiments of the system or computer readable media disclosed herein, the computer readable media include non-transitory computer-executable instructions which, when executed by the at least one electronic processor further perform at least: using the non-target nucleic acid variant filter list to determine whether a test sample obtained from a subject comprises cfNA fragments that originate from the non-target cells.

### BRIEF DESCRIPTION OF THE DRAWINGS

The accompanying drawings (also “Figure” and “FIG.” herein), which are incorporated in and constitute a part of this specification, illustrate certain embodiments, and together with the written description, serve to explain certain principles of the methods, computer readable media, and systems disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIGS. 1A and 1B are histograms of two alleles (FIG. 1A shows Classification Allele 1, while FIG. 1B shows Classification Allele 2) differentiated by subclonality score estimated based on a clonality border value set at a 50% threshold. Allele 1 is negative (i.e., not indicative of a cancer cell source (likely a hematopoietic stem cell)), whereas Allele 2 is positive as being present in more than 50% of subjects in a reference sample database (i.e., indicative of a cancer cell source) according to some embodiments of the invention. In each of FIGS. 1A and 1B, the Y-axis shows the number of records, while the X-axis shows MAF/maxMAF ratio distributions.

FIG. 2 is a flow chart that schematically depicts exemplary method steps of detecting a nucleic acid molecule that originates from a target cell in a subject according to some embodiments of the invention.

FIG. 3 is a flow chart that schematically depicts exemplary method steps of detecting a nucleic acid molecule that originates from a tumor cell in a subject according to some embodiments of the invention.

FIG. 4 is a flow chart that schematically depicts exemplary method steps of treating a disease in a subject according to some embodiments of the invention.

FIG. 5 is a flow chart that schematically depicts exemplary method steps of generating a classifier according to some embodiments of the invention.

FIG. 6 is a flow chart that schematically depicts exemplary method steps of generating a classifier according to some embodiments of the invention.

FIG. 7 is a schematic diagram of an exemplary system suitable for use with certain embodiments of the invention.

FIGS. 8A-C shows Kaplan-Meier plots for patient data using no filter (FIG. 8A), tissue filtering (FIG. 8B), and classifier filtering (FIG. 8C; i.e., used subclonality scores). The not detected curves are the upper curves, whereas the detected curves are the lower curves, in each of the plots shown in FIGS. 8A-C.

FIG. 9 shows a plot of allele frequency ranges (x-axis) versus the number of variants (y-axis) observed for the different filter scenarios depicted in FIGS. 8A-C.

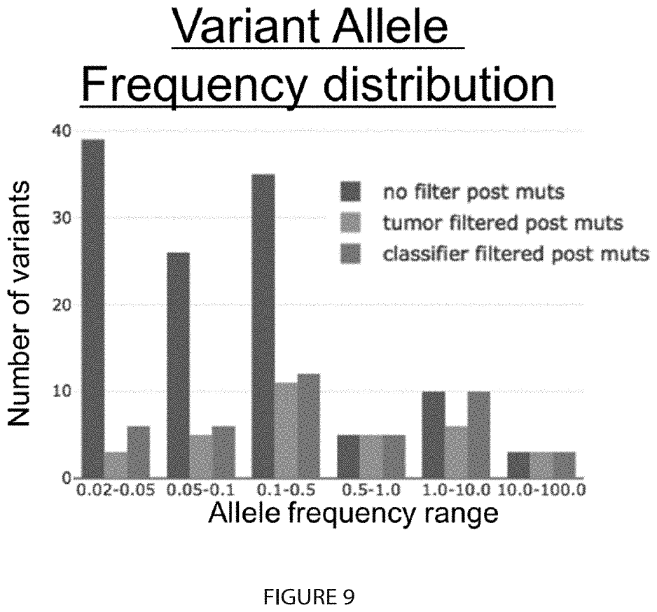

### DEFINITIONS

In order for the present disclosure to be more readily understood, certain terms are first defined below. Additional definitions for the following terms and other terms may be set forth through the specification. If a definition of a term set forth below is inconsistent with a definition in a patent application or issued patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons of ordinary skill in the art upon reading this disclosure and so forth. It will also be appreciated that there is an implied “about” prior to the temperatures, concentrations, times, number of bases or base pairs, coverage, etc. discussed in the present disclosure, such that slight and insubstantial equivalents are within the scope of the present disclosure. In this application, the use of the singular includes the plural unless specifically stated otherwise. Also, the use of “comprise”, “comprises”, “comprising”, “contain”, “contains”, “containing”, “include”, “includes”, and “including” are not intended to be limiting.

It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this disclosure pertains. In describing and claiming the methods, computer readable media, and systems, the following terminology, and grammatical variants thereof, will be used in accordance with the definitions set forth below.

About: As used herein, “about” or “approximately” as applied to one or more values or elements of interest, refers to a value or element that is similar to a stated reference value or element. In certain embodiments, the term “about” or “approximately” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Adapter: As used herein, “adapter” refers to short nucleic acids (e.g., less than about 500, less than about 100 or less than about 50 nucleotides in length) that are typically at least partially double-stranded and used to link to either or both ends of a given sample nucleic acid molecule. Adapters can include nucleic acid primer binding sites to permit amplification of a nucleic acid molecule flanked by adapters at both ends, and/or a sequencing primer binding site, including primer binding sites for sequencing applications, such as various next generation sequencing (NGS) applications. Adapters can also include binding sites for capture probes, such as an oligonucleotide attached to a flow cell support or the like. Adapters can also include a nucleic acid tag as described herein. Nucleic acid tags are typically positioned relative to amplification primer and sequencing primer binding sites, such that a nucleic acid tag is included in amplicons and sequencing reads of a given nucleic acid molecule. The same or different adapters can be linked to the respective ends of a nucleic acid molecule. In certain embodiments, the same adapter is linked to the respective ends of the nucleic acid molecule except that the nucleic acid tag differs. In some embodiments, the adapter is a Y-shaped adapter in which one end is blunt ended or tailed as described herein, for joining to a nucleic acid molecule, which is also blunt ended or tailed with one or more complementary nucleotides. In still other exemplary embodiments, an adapter is a bell-shaped adapter that includes a blunt or tailed end for joining to a nucleic acid molecule to be analyzed. Other exemplary adapters include T-tailed and C-tailed adapters.

Administer: As used herein, “administer” or “administering” a therapeutic agent (e.g., an immunological therapeutic agent) to a subject means to give, apply or bring the composition into contact with the subject. Administration can be accomplished by any of a number of routes, including, for example, topical, oral, subcutaneous, intramuscular, intraperitoneal, intravenous, intrathecal and intradermal.

Allele: As used herein, “allele” or “allelic variant” refers to a specific genetic variant at defined genomic location or locus. An allelic variant is usually presented at a frequency of 50% (0.5) or 100%, depending on whether the allele is heterozygous or homozygous. For example, germline variants are inherited and usually have a frequency of 0.5 or 1. Somatic variants; however, are acquired variants and usually have a frequency of <0.5. Major and minor alleles of a genetic locus refer to nucleic acids harboring the locus in which the locus is occupied by a nucleotide of a reference sequence, and a variant nucleotide different than the reference sequence respectively. Measurements at a locus can take the form of allelic fractions (AFs), which measure the frequency with which an allele is observed in a sample.

Amplify: As used herein, “amplify” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes.

Barcode: As used herein, “barcode” in the context of nucleic acids refers to a nucleic acid molecule comprising a sequence that can serve as a molecular identifier. For example, individual “barcode” sequences are typically added to each DNA fragment during next-generation sequencing (NGS) library preparation so that each read can be identified and sorted before the final data analysis.

Cancer Type: As used herein, “cancer,” “cancer type” or “tumor type” refers to a type or subtype of cancer defined, e.g., by histopathology. Cancer type can be defined by any conventional criterion, such as on the basis of occurrence in a given tissue (e.g., blood cancers, central nervous system (CNS), brain cancers, lung cancers (small cell and non-small cell), skin cancers, nose cancers, throat cancers, liver cancers, bone cancers, lymphomas, pancreatic cancers, bowel cancers, rectal cancers, thyroid cancers, bladder cancers, kidney cancers, mouth cancers, stomach cancers, breast cancers, prostate cancers, ovarian cancers, lung cancers, intestinal cancers, soft tissue cancers, neuroendocrine cancers, gastroesophageal cancers, head and neck cancers, gynecological cancers, colorectal cancers, urothelial cancers, solid state cancers, heterogeneous cancers, homogenous cancers), unknown primary origin and the like, and/or of the same cell lineage (e.g., carcinoma, sarcoma, lymphoma, cholangiocarcinoma, leukemia, mesothelioma, melanoma, or glioblastoma) and/or cancers exhibiting cancer markers, such as Her2, CA15-3, CA19-9, CA-125, CEA, AFP, PSA, HCG, hormone receptor and NMP-22. Cancers can also be classified by stage (e.g., stage 1, 2, 3, or 4) and whether of primary or secondary origin.

Cell-Free Nucleic Acid: As used herein, “cell-free nucleic acid” refers to nucleic acids not contained within or otherwise bound to a cell or, in some embodiments, nucleic acids remaining in a sample following the removal of intact cells. Cell-free nucleic acids can include, for example, all non-encapsulated nucleic acids sourced from a bodily fluid (e.g., blood, plasma, serum, urine, cerebrospinal fluid (CSF), etc.) from a subject. Cell-free nucleic acids include DNA (cfDNA), RNA (cfRNA), and hybrids thereof, including genomic DNA, mitochondrial DNA, circulating DNA, siRNA, miRNA, circulating RNA (cRNA), tRNA, rRNA, small nucleolar RNA (snoRNA), Piwi-interacting RNA (piRNA), long non-coding RNA (long ncRNA), and/or fragments of any of these. Cell-free nucleic acids can be double-stranded, single-stranded, or a hybrid thereof. A cell-free nucleic acid can be released into bodily fluid through secretion or cell death processes, e.g., cellular necrosis, apoptosis, or the like. Cell-free nucleic acids can be found in an efferosome or an exosome. Some cell-free nucleic acids are released into bodily fluid from cancer cells, e.g., circulating tumor DNA (ctDNA). Others are released from healthy cells. CtDNA can be non-encapsulated tumor-derived fragmented DNA. Another example of cell-free nucleic acids is fetal DNA circulating freely in the maternal blood stream, also called cell-free fetal DNA (cffDNA). A cell-free nucleic acid can have one or more epigenetic modifications, for example, a cell-free nucleic acid can be acetylated, 5-methylated, ubiquitylated, phosphorylated, sumoylated, ribosylated, and/or citrullinated.

Cellular Origin: As used herein, “cellular origin” in the context of cell-free nucleic acids means the cell type from which a given cell-free nucleic acid molecule derives or otherwise originates (e.g., via a apoptotic process, a necrotic process, or the like). In certain embodiments, for example, a given cell-free nucleic acid molecule may originate from a tumor cell (e.g., a cancerous pulmonary cell, etc.) or a non-tumor or normal cell (e.g., a non-cancerous pulmonary cell, a hematopoietic stem cell, etc.).

Classification Allele: As used herein, “classification allele” refers to an allelic variant the presence of which in a given nucleic acid molecule identifies the origin (e.g., cellular origin) of that nucleic acid molecule. In certain embodiments, for example, the presence of a given classification allele in a nucleic acid molecule may identify that nucleic acid molecule as originating from a target cell (e.g., a diseased cell, a tumor cell, a fetal cell, a transplant donor cell, or the like) or from a non-target cell (e.g., a non-diseased cell, a hematopoietic stem cell, a maternal cell, a transplant recipient cell, or the like) depending on the particular application. Typically, a classification allele is associated with a subclonality score that can be used to assign the given classification allele to a target or non-target nucleic acid variant filter list depending upon whether the subclonality score is below, or at or above, a selected cutoff threshold value used in a given application.

Classifier: As used herein, “classifier” generally refers to algorithm computer code that receives, as input, test data and produces, as output, a classification of the input data as belonging to one or another class (e.g., tumor DNA or non-tumor DNA).

Clinical Information: As used herein, “clinical information” refers any information that can inform health care decisions for a subject. Examples of clinical information, includes, but is not limited to, genomic information, age, gender, race, weight, body mass index (BMI), clinical history, drug usage, tobacco usage, and alcohol usage, among many others.

Clonal Hematopoiesis-derived Mutation: As used herein, “clonal hematopoiesis-derived mutation” refers to the somatic acquisition of genomic mutations in hematopoietic stem and/or progenitor cells leading to clonal expansion.

Clonal Hematopoiesis of Indeterminate Potential: As used herein, “clonal hematopoiesis of indeterminate potential” or “CHIP” refers to hematopoiesis in individuals that involves the expansion of hematopoietic stem cells that comprise one or more somatic mutations (e.g., hematologic malignancy-associated mutations and/or not), but which otherwise lack diagnostic criteria for a hematologic malignancy, such as definitive morphologic evidence of dysplasia. CHIP is a common age-related phenomenon in which hematopoietic stem cells contribute to the formation of a genetically distinct subpopulation of blood cells.

Clonality Border Value: As used herein, “clonality border value” refers to a selected value used in the calculation of a given subclonality score.

Comparator Result: As used herein, “comparator result” or “reference result” means a result or set of results to which a given test sample or test result can be compared to identify one or more likely properties of the test sample or result, and/or one or more possible prognostic outcomes and/or one or more customized therapies for the subject from whom the test sample was taken or otherwise derived. Comparator results are typically obtained from a set of reference samples (e.g., from subjects having the same disease or cancer type as the test subject and/or from subjects who are receiving, or who have received, the same therapy as the test subject).

Control Sample: As used herein, “control sample” or “control DNA sample” refers a sample of known composition and/or having known properties and/or known parameters (e.g., known cellular origin, known tumor fraction, known coverage, and/or the like) that is analyzed along with or compared to test samples in order to evaluate the accuracy of an analytical procedure. A control sample dataset typically includes from at least about 25 to at least about 30,000 or more control samples. In some embodiments, the control sample dataset includes about 50, 75, 100, 150, 200, 300, 400, 500, 600, 700, 800, 900, 1,000, 2,500, 5,000, 7,500, 10,000, 15,000, 20,000, 25,000, 50,000, 100,000, 1,000,000, or more control samples.

Coverage: As used herein, “coverage” refers to the number of nucleic acid molecules that represent a particular base position.

Cutoff Threshold Value: As used herein, “cutoff threshold value” refers to a selected value to which a subclonality score is compared in order to assign a classification allele having that subclonality score to a target nucleic acid variant filter list or to a non-target nucleic acid variant filter list.

Deoxyribonucleic Acid or Ribonucleic Acid: As used herein, “deoxyribonucleic acid” or “DNA” refers a natural or modified nucleotide which has a hydrogen group at the 2′-position of the sugar moiety. DNA typically includes a chain of nucleotides comprising deoxyribonucleosides that each comprise one of four types of nucleobases, namely, adenine (A), thymine (T), cytosine (C), and guanine (G). As used herein, “ribonucleic acid” or “RNA” refers to a natural or modified nucleotide which has a hydroxyl group at the 2′-position of the sugar moiety. RNA typically includes a chain of nucleotides comprising ribonucleosides that each comprise one of four types of nucleobases, namely, A, uracil (U), G, and C. As used herein, the term “nucleotide” refers to a natural nucleotide or a modified nucleotide. Certain pairs of nucleotides specifically bind to one another in a complementary fashion (called complementary base pairing). In DNA, adenine (A) pairs with thymine (T) and cytosine (C) pairs with guanine (G). In RNA, adenine (A) pairs with uracil (U) and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “sequence information,” “nucleic acid sequence,” “nucleotide sequence”, “genomic sequence,” “genetic sequence,” or “fragment sequence,” or “nucleic acid sequencing read” denotes any information or data that is indicative of the order and identity of the nucleotide bases (e.g., adenine, guanine, cytosine, and thymine or uracil) in a molecule (e.g., a whole genome, whole transcriptome, exome, oligonucleotide, polynucleotide, or fragment) of a nucleic acid such as DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, and electronic signature-based systems.

Fragment: As used herein, “fragment” in the context of cell-free nucleic acids refers to a nucleic acid molecule that is naturally present in the body of a subject (or in a sample obtained from the subject), and should not be construed as requiring a fragmentation step be performed in vitro.

Hematopoietic Stem Cell: As used herein, “hematopoietic stem cell” or “HSC” is a stem cell that gives rise to other blood cells through the process of haematopoiesis.

Immunotherapy: As used herein, “immunotherapy” refers to treatment with one or more agents that act to stimulate the immune system so as to kill or at least to inhibit growth of cancer cells, and preferably to reduce further growth of the cancer, reduce the size of the cancer and/or eliminate the cancer. Some such agents bind to a target present on cancer cells; some bind to a target present on immune cells and not on cancer cells; some bind to a target present on both cancer cells and immune cells. Such agents include, but are not limited to, checkpoint inhibitors and/or antibodies. Checkpoint inhibitors are inhibitors of pathways of the immune system that maintain self-tolerance and modulate the duration and amplitude of physiological immune responses in peripheral tissues to minimize collateral tissue damage (see, e.g., Pardoll, Nature Reviews Cancer 12, 252-264 (2012)). Exemplary agents include antibodies against any of PD-1, PD-2, PD-L1, PD-L2, CTLA-40, OX40, B7.1, B7He, LAG3, CD137, KIR, CCR5, CD27, or CD40. Other exemplary agents include proinflammatory cytokines, such as IL-1β, IL-6, and TNF-α. Other exemplary agents are T-cells activated against a tumor, such as T-cells activated by expressing a chimeric antigen targeting a tumor antigen recognized by the T-cell.

Indel: As used herein, “indel” refers to mutation that involves the insertion or deletion of nucleotide positions in the genome of a subject.

Indexed: As used herein, “indexed” refers to a first element (e.g., clinical information) linked to a second element (e.g., a given sample).

Maximum Minor Allele Frequency: As used herein, “maximum minor allele frequency,” “maximum MAF,” or “maxMAF” refers to the maximum or largest MAF of all somatic variants present or observed in a given sample.

Minor Allele Frequency: As used herein, “minor allele frequency” or “MAF” refers to the frequency at which minor alleles (e.g., not the most common allele) occur in a given population of nucleic acids, such as a sample obtained from a subject. In other words, “minor allele frequency” means the frequency of an allele observed at a given locus in a given sample that is not the most prevalent allele observed at that locus in that sample. MAF is generally expressed as a fraction or a percentage. For example, an MAF is typically less than about 0.5, 0.1, 0.05, or 0.01 (i.e., less than about 50%, 10%, 5%, or 1%) of all somatic variants or alleles present at a given locus.

Mutation: As used herein, “mutation” or “genetic aberration” refers to a variation from a known reference sequence and includes mutations such as, for example, single nucleotide variants (SNVs), copy number variants or variations (CNVs)/aberrations, insertions or deletions (indels), truncation, gene fusions, transversions, translocations, frame shifts, duplications, repeat expansions, and epigenetic variants. A mutation can be a germline or somatic mutation. In some embodiments, a reference sequence for purposes of comparison is a wildtype genomic sequence of the species of the subject providing a test sample, typically the human genome.

Neoplasm: As used herein, the terms “neoplasm” and “tumor” are used interchangeably. They refer to abnormal growth of cells in a subject. A neoplasm or tumor can be benign, potentially malignant, or malignant. A malignant tumor is a referred to as a cancer or a cancerous tumor.

Next Generation Sequencing: As used herein, “next generation sequencing” or “NGS” refers to sequencing technologies having increased throughput as compared to traditional Sanger- and capillary electrophoresis-based approaches, for example, with the ability to generate hundreds of thousands of relatively small sequence reads at a time. Some examples of next generation sequencing techniques include, but are not limited to, sequencing by synthesis, sequencing by ligation, and sequencing by hybridization.

Nucleic Acid Tag: As used herein, “nucleic acid tag” refers to a short nucleic acid (e.g., less than about 500, about 100, about 50 or about 10 nucleotides in length), used to label nucleic acid molecules to distinguish nucleic acids from different samples (e.g., representing a sample index), or different nucleic acid molecules in the same sample (e.g., representing a molecular tag), of different types, or which have undergone different processing. Nucleic acid tags can be single stranded, double stranded or at least partially double stranded. Nucleic acid tags optionally have the same length or varied lengths. Nucleic acid tags can also include double-stranded molecules having one or more blunt-ends, include 5′ or 3′ single-stranded regions (e.g., an overhang), and/or include one or more other single-stranded regions at other locations within a given molecule. Nucleic acid tags can be attached to one end or both ends of the other nucleic acids (e.g., sample nucleic acids to be amplified and/or sequenced). Nucleic acid tags can be decoded to reveal information such as the sample of origin, form or processing of a given nucleic acid. Nucleic acid tags can also be used to enable pooling and/or parallel processing of multiple samples comprising nucleic acids bearing different nucleic acid tags and/or sample indexes in which the nucleic acids are subsequently being deconvoluted by reading the nucleic acid tags. Nucleic acid tags can also be referred to as molecular identifiers or tags, sample identifiers, index tags, and/or barcodes. Additionally or alternatively, nucleic acid tags can be used to distinguish different molecules in the same sample. This includes, for example, uniquely tagging each different nucleic acid molecule in a given sample, or non-uniquely tagging such molecules. In the case of non-unique tagging applications, a limited number of tags may be used to tag each nucleic acid molecule such that different molecules can be distinguished based on, for example, start/stop positions where they map to a selected reference genome in combination with at least one nucleic acid tag. Typically, a sufficient number of different nucleic acid tags are used such that there is a low probability (e.g., less than about a 10%, less than about a 5%, less than about a 1%, or less than about a 0.1% chance) that any two molecules will have the same start/stop positions and also have the same nucleic acid tag. Some nucleic acid tags include multiple molecular identifiers to label samples, forms of nucleic acid molecules within a sample, and nucleic acid molecules within a form having the same start and stop positions. Such nucleic acid tags can be referenced using the exemplary form “A1i” in which the uppercase letter indicates a sample type, the Arabic numeral indicates a form of molecule within a sample, and the lowercase Roman numeral indicates a molecule within a form.

Polynucleotide: As used herein, “polynucleotide”, “nucleic acid”, “nucleic acid molecule”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Oligonucleotides often range in size from a few monomeric units, e.g. 3-4, to hundreds of monomeric units. Whenever a polynucleotide is represented by a sequence of letters, such as “ATGCCTG,” it will be understood that the nucleotides are in 5′→3′ order from left to right and that in the case of DNA, “A” denotes deoxyadenosine, “C” denotes deoxycytidine, “G” denotes deoxyguanosine, and “T” denotes deoxythymidine, unless otherwise noted. The letters A, C, G, and T may be used to refer to the bases themselves, to nucleosides, or to nucleotides comprising the bases, as is standard in the art

Potential Clinical Significance: As used herein, “potential clinical significance” in the context of allelic variants refers to an allele the presence of which in a given nucleic acid molecule from a subject may inform health care decisions for that subject.

Reference Sequence: As used herein, “reference sequence” or “reference genome” refers to a known sequence used for purposes of comparison with experimentally determined sequences. For example, a known sequence can be an entire genome, a chromosome, or any segment thereof. A reference sequence typically includes at least about 20, at least about 50, at least about 100, at least about 200, at least about 250, at least about 300, at least about 350, at least about 400, at least about 450, at least about 500, at least about 1000, or more nucleotides. A reference sequence can align with a single contiguous sequence of a genome or chromosome or can include non-contiguous segments that align with different regions of a genome or chromosome. Exemplary reference sequences, include, for example, human genomes, such as, hG19 and hG38.

Sample: As used herein, “sample” means anything capable of being analyzed by the methods and/or systems disclosed herein.

Sensitivity: As used herein, “sensitivity” in the context of a given assay or method refers to the ability of the assay or method to detect and distinguish between targeted (e.g., cfDNA fragments originating from tumor cells) and non-targeted (e.g., cfDNA fragments originating from non-tumor cells) analytes.

Sequencing: As used herein, “sequencing” refers to any of a number of technologies used to determine the sequence (e.g., the identity and order of monomer units) of a biomolecule, e.g., a nucleic acid such as DNA or RNA. Exemplary sequencing methods include, but are not limited to, targeted sequencing, single molecule real-time sequencing, exon or exome sequencing, intron sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, and a combination thereof. In some embodiments, sequencing can be performer by a gene analyzer such as, for example, gene analyzers commercially available from Illumina, Inc., Pacific Biosciences, Inc., or Applied Biosystems/Thermo Fisher Scientific, among many others.

Sequence Information: As used herein, “sequence information” in the context of a nucleic acid polymer means the order and identity of monomer units (e.g., nucleotides, etc.) in that polymer.

Somatic Mutation: As used herein, “somatic mutation” means a mutation in the genome that occurs after conception. Somatic mutations can occur in any cell of the body except germ cells and accordingly, are not passed on to progeny.

Splice Site Variant: As used herein, “splice site variant” in the context of nucleic acid mutations refers to a genetic alteration in a given DNA sequence that occurs at the boundary of an exon and an intron (splice site). This change can disrupt RNA splicing resulting in the loss of exons or the inclusion of introns and an altered protein-coding sequence.

Specificity: As used herein, “specificity” in the context of a diagnostic analysis or assay refers to the extent to which the analysis or assay detects an intended target analyte to the exclusion of other components of a given sample.

Subclonality Score: As used herein, “subclonality score” is a ratio of the number of times a given allele is observed to have a MAF/maxMAF ratio value below a clonality border value in a set of samples over (i.e., divided by) the total number of times that given allele is observed or occurred in that set of samples.

Subject: As used herein, “subject” or “test subject” refers to an animal, such as a mammalian species (e.g., human) or avian (e.g., bird) species, or other organism, such as a plant. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual that is in need of therapy or suspected of needing therapy. The terms “individual” or “patient” are intended to be interchangeable with “subject.” In some embodiments, the subject is a human who has, or is suspected of having cancer. For example, a subject can be an individual who has been diagnosed with having a cancer, is going to receive a cancer therapy, and/or has received at least one cancer therapy. The subject can be in remission of a cancer. As another example, the subject can be an individual who is diagnosed of having an autoimmune disease. As another example, the subject can be a female individual who is pregnant or who is planning on getting pregnant, who may have been diagnosed with or suspected of having a disease, e.g., a cancer, an auto-immune disease.

Substantial Match: As used herein, “substantial match” means that at least a first value or element is at least approximately equal to at least a second value or element. In certain embodiments, for example, the cellular origin of a given allelic variant from a cfDNA sample is determined when there is at least a substantial or approximate match (e.g., a sequence alignment and/or other clinical information or properties) between that allelic variant and a reference sample or classification allele.

Substantially Align: As used herein, the phrase “substantially align” in the context of nucleic acid sequence alignment means that a first nucleic acid sequence has at least 95%, at least 96%, at least 97%, at least 98%, at least 99%, or even 100% sequence identity with at least a sub-sequence of a second nucleic acid sequence. In some embodiments, for example, a given sequence read substantially aligns with a reference sequence when the given sequence read has 95%, 96%, 97%, 98%, 99%, or 100% sequence identity with at least a sub-sequence or region, or the entirety, of the reference sequence.

Threshold: As used herein, “threshold” refers to a separately determined value used to characterize or classify experimentally determined values.

Truncation: As used herein, “truncation” in the context of nucleic acid mutations refers to sequence variation observed in a given DNA sequence that can truncate or shorten a polypeptide (e.g., a protein) encoded by that DNA sequence upon expression.

Tumor Fraction: As used herein, “tumor fraction” refers to the estimate of the fraction of nucleic acid molecules derived from tumor in a given sample. For example, the tumor fraction of a sample can be a measure derived from the maximum minor allele frequency (maxMAF) of the sample or coverage of the sample, or length, epigenetic state, or other properties of the cfDNA fragments in the sample or any other selected feature of the sample. The term “maxMAF” refers to the maximum or largest MAF of all somatic variants present in a given sample. In some embodiments, the tumor fraction of a sample is equal to the maxMAF of the sample.

Value: As used herein, “value” generally refers to an entry in a dataset can be anything that characterizes the feature to which the value refers. This includes, without limitation, numbers, words or phrases, symbols (e.g., + or −) or degrees.

### DETAILED DESCRIPTION

### Introduction

Provided herein are methods, computer readable media, and systems for producing improved sensitivity and/or specificity of detecting cancer cell DNA or other target nucleic acids in cell free nucleic acid (cfNA) present in samples obtained from patients. The subject methods, computer readable media, and systems may be readily applied to cfDNA analysis of tumor and other target cfNAs, such as the techniques described in U.S. Pat. No. 9,920,366 B2, U.S. Pat. No. 9,840,743 B2, and PCT published patent application WO 2017/181146 A1, which are each incorporated by reference. In some embodiments, provided herein are methods of identifying alleles that can be used to determine if they originated from a cancer cell or a hematopoietic stem cell. Once identified, such informative alleles can be used to classify a sample as containing tumor cell DNA or not containing tumor cell DNA in certain exemplary embodiments.

Methods and Related Aspects of Determining the Cellular Origin of cfNA

This application discloses various methods related to determining whether cell-free nucleic acids (cfNA) samples comprise nucleic acid molecules or fragments originating from given cell- or tissue-types. In some exemplary embodiments, the methods are used to determine whether a cfNA sample includes nucleic acid molecules (e.g., cell-free deoxyribonucleic acid (cfDNA) fragments and/or cell-free ribonucleic acid (cfRNA) fragments), originating from diseased cells (e.g., tumor cells, or the like), fetal cells, transplant donor cells, and/or the like. Frequently, these types of nucleic acid molecules represent only a small fraction of all nucleic acid molecules present in a given cfNA sample, which generally includes a large background of nucleic acid molecules originating from, for example, non-diseased, normal, or healthy cells (e.g., hematopoietic stem cells or other non-tumor cells), maternal cells, transplant recipient cells, and/or the like. Many pre-existing analytical techniques lack sufficient sensitivity to reliably detect and characterize nucleic acid molecules present in such low numbers in cfNA samples. The information obtained from the methods disclosed herein is typically used to diagnose whether a subject from whom the cfNA sample was obtained has a given disease, disorder, or condition. In certain embodiments, the methods include administering therapy or otherwise treating the diagnosed disease, disorder, or condition in subjects. This application also discloses, for example, related methods of generating classifiers as well as methods of producing databases of subclonality scores of use in classifying the cellular origin of cfNA fragments in test samples.

In various embodiments of the subject methods, a plurality of loci are sequenced so as to detect the allelic variants of the loci and the allele frequency at each of those loci. The DNA can come from a variety of cellular sources each producing cell free DNA, thereby producing a mixture of cell free DNA derived from different genomic sources for the same locus. The DNA source may be a tumor cell, including several different clonally different tumor cell variants present in the same subject, and non-tumor cell, especially blood cells. In some embodiments regions of the genome are targeted for sequencing (in contrast to whole genome sequencing). By using high throughput DNA sequencers multiple fragments of cfDNA from the sample may be concurrently sequenced so as to detect multiple alleles at the same locus and provide for the allele frequency at that locus. Clonal hematopoiesis of indeterminate potential (CHIP) is a common age-related phenomenon in which hematopoietic stem cells contribute to the formation of a genetically distinct subpopulation of blood cells. These hematopoietic stem cells can produce cell free DNA allelic information that may be confused with the allelic variants produced in cancerous cells.

Databases of allelic information from reference subjects may be used to discover alleles that can be used to classify a cell free DNA sample as containing tumor cell DNA or not. The databases typically comprises cell free DNA sequence information from any subjects suspected of having cancer. In general, the larger the database, the more useful the database is for identifying allelic variants that can be used to discover allelic variants that are indicative of the presence or absence of tumor cell DNA in the cell free DNA. Multiple genetic loci of potential clinical significance are sequenced for each patient in the database, and for each locus sequenced, the frequency of each allele at the locus is determined. The minor allele frequency (MAF) is determined for each locus. Because of genetic heterogeneity in a given cfDNA sample, each MAF may vary significantly between loci. For example, a driver mutation at a locus is likely to have a higher MAF than a passenger mutation that is acquired in later clones during evolution of the tumor. For a given patient, the allele among the set of analyzed alleles having the maximum MAF (maxMAF) is determined and the value for the MAF of the maxMAF is also determined. The database may also include other clinical information for each patient, so that the other clinical information can be correlated with the genetic information for each patient. Examples of such clinical information include tumor detection, patient survival, patient age, and the like.

The allelic information in the database can then be screened for alleles that that can be used to classify a cfDNA sample as either comprising tumor DNA of clinical significance or not comprising tumor DNA of clinical significance. For each given allelic variant of potential clinical significance in a test sample, the ratio of the minor allele frequency (MAF) to the maxMAF is determined. An MAF/maxMAF ratio calculation is then typically created for many samples in the database for the allele of interest. The frequency of each MAF/maxMAF value for a given allele within the database (or portion of the database) can then be determined. For example, a histogram of the MAF/maxMAF values may be plotted. A clonality border value can then be set for computing a subclonality score, which is a ratio of number of cases when a given allele within the database has MAF/maxMAF value below the clonality border over the total number of cases when the given allele has been observed in samples represented in the database. A cutoff threshold can then be set for deciding whether or not a given allele is indicative of the presence of tumor DNA. Alleles with subclonality scores above the threshold can be used to identify alleles that come from non-tumor DNA. Conversely, alleles with subclonality scores below the threshold can be used to identify alleles that come from tumor DNA.

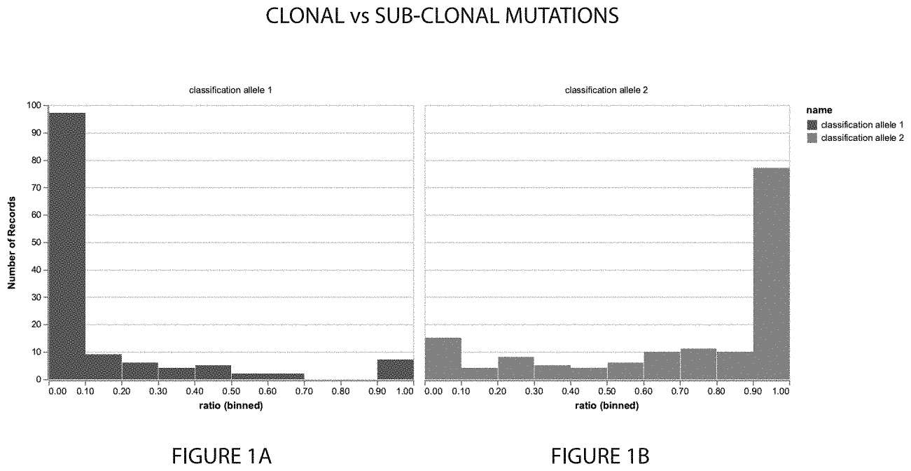

To illustrate, a 50% clonality border value could be set as shown, for example, in FIGS. 1 A and B. As shown, Allele 1 has a high subclonality score estimated based on the clonality border value set at 50%, while Allele 2 has a low subclonality score. Thus, alleles can, in some embodiments, fall into one of the two categories, indicative of non-tumor DNA (negative, e.g., Allele 1 in FIG. 1A) or, indicative of tumor DNA (positive, e.g., Allele 2 in FIG. 1B). In the example provided in FIGS. 1 A and B, Allele 1 and Allele 2 are present in different genes, i.e., not variant alleles of the same locus. This analysis can be applied to multiple tested alleles in the database, putting a given allele in either the positive or negative category, thereby producing sets of positive and negative alleles. Optionally, more stringent selection thresholds may be applied so as to exclude alleles from either category, thus not using them to make a classification decision for a given sample. For example, alleles with subclonality scores below 25% could be positive and alleles with subclonality scores above 75% could be negative, while those alleles in the excluded range (i.e., from 25% and 75%) are not used to classify the samples as containing or not containing tumor DNA. The categorized alleles can be used to produce a list of alleles to classify a given test sample obtained from a subject. Such a list is referred to as a “tumor variant filter list” or a “non-tumor variant filter list, depending on the context of the usage of the term. Again, FIGS. 1 A and B provide an example in which the clonality border value for MAF/maxMAF distributions is set at 50% for the samples represented in the database.

A low subclonality score typically suggests that an observed allele is indicative of the presence of tumor DNA. For example, a score of zero would indicate that in every sample represented within the database where the allele was observed, the MAF/maxMAF is greater than the clonality border value, which would indicate that the allele has been the dominant minor allele in every sample within the database.

Other classification criteria in addition to test for the presence or absence of positive (i.e., of tumor origin) and negative (i.e., of non-tumor origin) alleles in a sample are also optionally used. The use of the information in positive and negative allele sets discovered from the MAF/maxMAF ratios to call clinically significant mutations is generally most useful for alleles having a low MAF. In some embodiments, for example, if a given variant allele is found to have an MAF of greater than 1% and the allele is a negative allele, the sample is still classified as containing tumor DNA, even if the allele is on the negative allele list. In another example, if a variant allele is found to have an MAF of greater than 2%, the sample is classified as containing tumor DNA, even if the allele is on the negative allele list in certain embodiments. In other embodiments, higher MAF thresholds may be used.

Another exemplary classification criterion is the type of allelic variant observed. In some embodiments, for example, an allelic variant is called as having clinical significance even if the variant is classified as negative by the subclonality score and the MAF is below a selected value (e.g., below 2% in some embodiments, below 1% in other embodiments). Allelic variants, such as truncations, indels, or splice site variants, are indicative of cancer and typically not present in hematopoietic stem cells.

In some embodiments, a cfDNA sample from a patient may be characterized as a containing cancer cell derived DNA if it meets any one of the following criteria: (1) having an allelic variant that is a truncation, indel, or splice site variant, (2) having a subclonality score positive allele, or (3) having a subclonality score negative allele with an MAF of greater than 1%. In some embodiments, a cfDNA sample from a patient may be characterized as a containing cancer cell derived DNA if it meets any one of the following criteria: (1) having an allelic variant that is a truncation, indel, or splice site variant, (2) having a subclonality score positive allele, or (3) having a subclonality score negative allele with an MAF of greater than 2%.

The frequency of CHIP mutations typically increases with patient age, accordingly the classification can make use of patient age and/or other patient data to determine if the cell free DNA sample contains tumor DNA in certain embodiments. In other exemplary embodiments, subclonal lists (e.g., target or non-target nucleic acid variant filter lists) are generated over different subsets of samples based on, for example, minimal maxMAF, calling maxMAF based on known driver mutations, and/or the like. In some embodiments, subclonal lists are generated based specific indications, such as a given cancer-type (e.g., lung, colorectal, etc.). In certain embodiments, machine learning classifiers are trained based upon one or more features, including mutant allele frequency, subclonal ratio, gene type, variants associated with hematological malignancies, patient age, observation of other CHIP variants, cancer type, and/or the like.

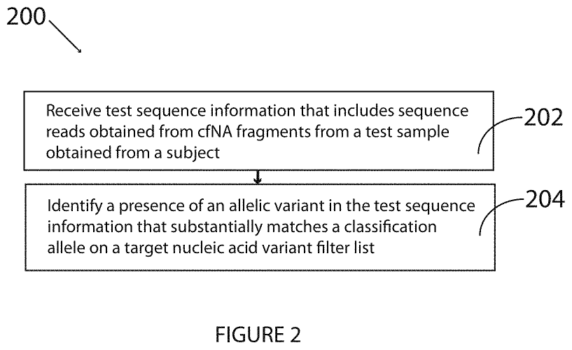

To further illustrate aspects of the methods disclosed herein, FIG. 2 provides a flow chart that schematically depicts exemplary method steps for detecting a nucleic acid molecule that originates from a target cell (e.g., a tumor cell or the like) in a subject at least partially using a computer. As shown, method 200 includes receiving, by the computer, test sequence information comprising sequence reads obtained from cell-free nucleic acid (cfNA) fragments from a test sample obtained from the subject in step 202. Method 200 also includes identifying a presence of at least one allelic variant in the test sequence information that substantially matches at least one classification allele on a target nucleic acid variant filter list, which classification allele comprises a subclonality score below at least one selected cutoff threshold value (e.g., about 10%, about 20%, about 30%, about 40%, about 50%, about 60%, about 70%, about 80%, about 90%, or another value) thereby indicating that the classification allele is from a reference cfNA fragment that originates from the target cell, thereby detecting the nucleic acid molecule that originates from the target cell in the subject in step 204. In some embodiments, for example, step 204 includes identifying at least one allelic variant in the test sequence information; mapping the allelic variant to at least one classification allele on a target nucleic acid variant filter list; identifying a subclonality score of the classification allele; and comparing the subclonality score to at least one selected cutoff threshold value in which when the subclonality score is below the selected cutoff threshold value it indicates that the classification allele is from a reference cfNA fragment that originates from the target cell. Related systems comprising computers and computer readable media are described further herein.

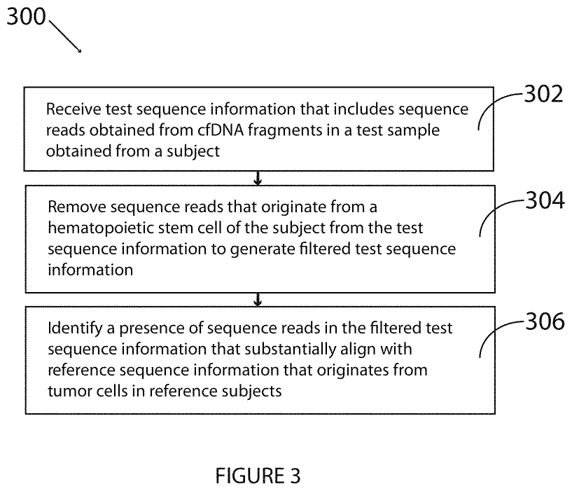

FIG. 3 provides a flow chart that schematically depicts exemplary method steps for detecting a nucleic acid molecule that originates from a tumor cell in a subject at least partially using a computer according to some embodiments. As shown, method 300 includes receiving, by the computer, test sequence information comprising sequence reads obtained from cell-free deoxyribonucleic acid (cfDNA) fragments in a test sample obtained from the subject in step 302. Method 300 also includes removing (e.g., deleting, suppressing, ignoring, or the like), by the computer, one or more of the sequence reads (e.g., that comprise at least portions of classification alleles) that originate from a hematopoietic stem cell of the subject from the test sequence information to generate filtered test sequence information in step 304. Method 300 additionally includes identifying, by the computer, a presence of one or more of the sequence reads in the filtered test sequence information that substantially align with reference sequence information obtained from one or more reference subjects, which reference sequence information originates from one or more tumor cells in the reference subjects, thereby detecting the nucleic acid molecule that originates from the tumor cell in the subject in step 306.

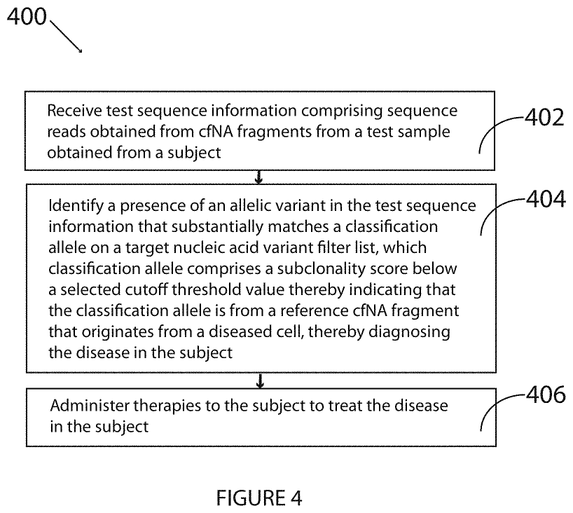

FIG. 4 provides a flow chart that schematically depicts exemplary method steps of treating a disease in a subject. As shown, method 400 includes receiving test sequence information comprising sequence reads obtained from cell-free nucleic acid (cfNA) fragments from a test sample obtained from the subject in step 402. Method 400 further includes identifying a presence of at least one allelic variant in the test sequence information that substantially matches at least one classification allele on a target nucleic acid variant filter list, which classification allele comprises a subclonality score below at least one selected cutoff threshold value thereby indicating that the classification allele is from a reference cfNA fragment that originates from a diseased cell, thereby diagnosing the disease in the subject in step 404. In addition, method 400 also includes administering one or more therapies to the subject, thereby treating the disease in the subject in step 406. Exemplary therapies are described further herein.

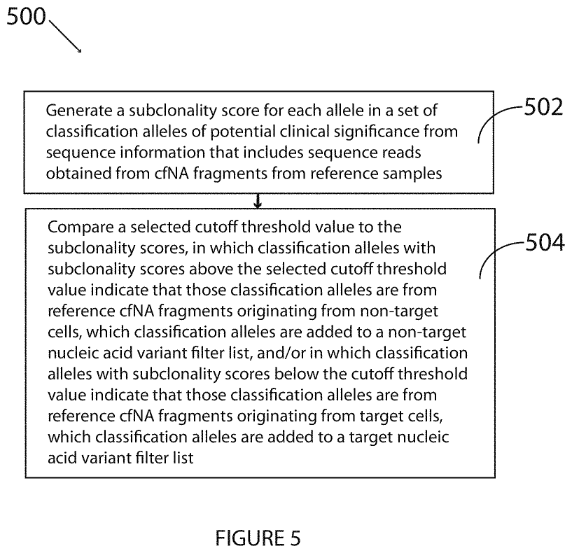

FIG. 5 provides a flow chart that schematically depicts exemplary method steps of generating a classifier at least partially using a computer. As shown, method 500 includes generating, by the computer, a subclonality score for each allele in a set of classification alleles from sequence information comprising sequence reads obtained from cell-free nucleic acid (cfNA) fragments from one or more reference samples, wherein each classification allele is of potential clinical significance and comprises a minor allele observed at a given locus in the reference samples in step 502. Method 500 also includes comparing, by the computer, at least one selected cutoff threshold value (e.g., about 10%, about 20%, about 30%, about 40%, about 50%, about 60%, about 70%, about 80%, about 90%, or another value) to the subclonality scores, in which classification alleles with subclonality scores above the selected cutoff threshold value indicate that those classification alleles are from reference cfNA fragments originating from non-target cells, which classification alleles are added to a non-target nucleic acid variant filter list, and/or in which classification alleles with subclonality scores below the cutoff threshold value indicate that those classification alleles are from reference cfNA fragments originating from target cells, which classification alleles are added to a target nucleic acid variant filter list, thereby generating the classifier in step 504.

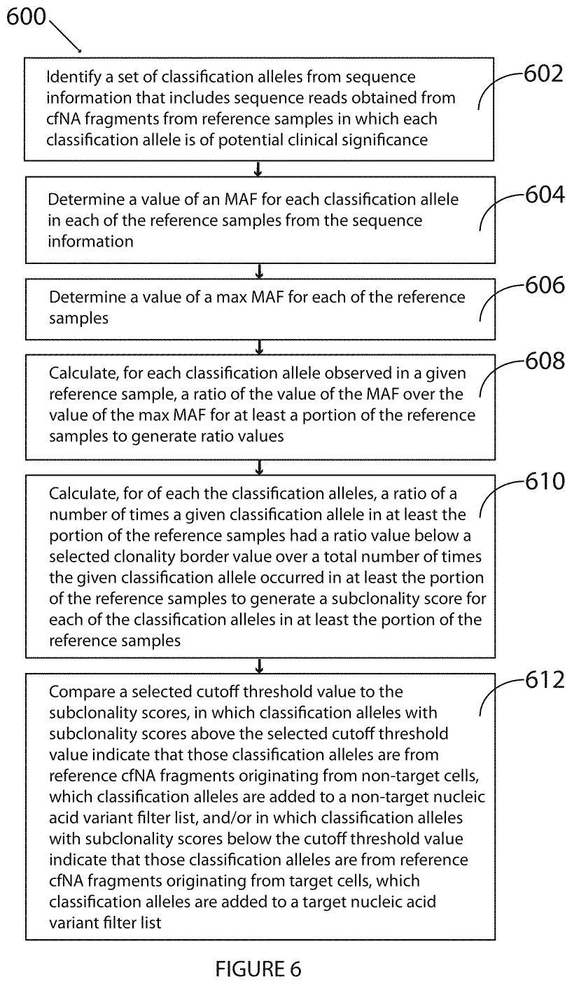

FIG. 6 provides a flow chart that schematically depicts exemplary method steps of generating a classifier at least partially using a computer. As shown, method 600 includes identifying, by the computer, a set of classification alleles from sequence information comprising sequence reads obtained from cell-free nucleic acid (cfNA) fragments from one or more reference samples, wherein each classification allele is of potential clinical significance and comprises a minor allele observed at a given locus in the reference samples in step 602. Method 600 also includes determining, by the computer, a value of a minor allele frequency (MAF) for each classification allele in each of the reference samples from the sequence information in step 604, and determining, by the computer, a value of a maximum minor allele frequency (maxMAF) for each of the reference samples in step 606. Method 600 also includes calculating, by the computer, for each classification allele observed in a given reference sample, a ratio of the value of the MAF over the value of the maxMAF for at least a portion of the reference samples to generate ratio values in step 608. Method 600 also includes calculating, by the computer, for of each the classification alleles, a ratio of a number of times a given classification allele in at least the portion of the reference samples had a ratio value below at least one selected clonality border value (e.g., about 10%, about 20%, about 30%, about 40%, about 50%, about 60%, about 70%, about 80%, about 90%, or another value) over a total number of times the given classification allele occurred in at least the portion of the reference samples to generate a subclonality score for each of the classification alleles in at least the portion of the reference samples in step 610. In addition, method 600 also includes comparing, by the computer, at least one selected cutoff threshold value (e.g., about 10%, about 20%, about 30%, about 40%, about 50%, about 60%, about 70%, about 80%, about 90%, or another value) to the subclonality scores, in which classification alleles with subclonality scores above the selected cutoff threshold value indicate that those classification alleles are from reference cfNA fragments originating from non-target cells, which classification alleles are added to a non-target nucleic acid variant filter list, and/or in which classification alleles with subclonality scores below the cutoff threshold value indicate that those classification alleles are from reference cfNA fragments originating from target cells, which classification alleles are added to a target nucleic acid variant filter list, thereby generating the classifier in step 612.

In some embodiments, the methods include obtaining the cfDNA sample from a subject. Essentially any sample type is optionally utilized. In certain embodiments, for example, the cfDNA sample is blood, plasma, serum, sputum, urine, semen, vaginal fluid, feces, synovial fluid, spinal fluid, saliva, and/or the like. Additional exemplary sample types that are optionally utilized are described further herein. Typically, the subject is a mammalian subject (e.g., a human subject). Essentially any type of nucleic acid (e.g., DNA and/or RNA) can be evaluated according to the methods disclosed in this application. Some examples, include cell-free nucleic acids (e.g., cfDNA of tumor origin, fetal origin, maternal origin, and/or the like), cellular nucleic acids, including circulating tumor cells (e.g., obtained by lysing intact cells in a sample), circulating tumor nucleic acids, and the like.

The methods disclosed in this application generally include obtaining sequence information from nucleic acids in samples taken from subjects. In certain embodiments, the sequence information is obtained from targeted segments of the nucleic acids. Essentially any number of genomic regions are optionally targeted. The targeted segments can include at least 10, at least 50, at least 100, at least 500, at least 1000, at least 2000, at least 5000, at least 10,000, at least 20,000 or at least 50, 000 (e.g., 25, 50, 75, 100, 200, 300, 400, 500, 600, 700, 800, 900, 1,000, 2,000, 3,000, 4,000, 5,000, 6,000, 7,000, 8,000, 9,000, 10,000, 15,000, 25,000, 30,000, 35,000, 40,000, 45,000) different and/or overlapping genomic regions.

In these embodiments, the methods also typically include various sample or library preparation steps to prepare nucleic acids for sequencing. Many different sample preparation techniques are well-known to persons skilled in the art. Essentially any of those techniques are used, or adapted for use, in performing the methods described herein. For example, in addition to various purification steps to isolate nucleic acids from other components in a given sample, typical steps to prepare nucleic acids for sequencing include tagging nucleic acids with molecular identifiers or barcodes, adding adapters (e.g., which may include the barcodes), amplifying the nucleic acids one or more times, enriching for targeted segments of the nucleic acids (e.g., using various target capturing strategies, etc.), and/or the like. Exemplary library preparation processes are described further herein. Additional details regarding nucleic acid sample/library preparation are also described in, for example, van Dijk et al., Library preparation methods for next-generation sequencing: Tone down the bias, Experimental Cell Research, 322(1):12-20 (2014), Micic (Ed.), Sample Preparation Techniques for Soil, Plant, and Animal Samples (Springer Protocols Handbooks), 1^{st} Ed., Humana Press (2016), and Chiu, Next-Generation Sequencing and Sequence Data Analysis, Bentham Science Publishers (2018), which are each incorporated by reference in their entirety.

The methods disclosed herein are typically used to diagnose the presence of a disease, disorder, or condition, particularly cancer, in a subject, to characterize such a disease, disorder, or condition (e.g., to stage a given cancer, to determine the heterogeneity of a cancer, and the like), to monitor response to treatment, to evaluate the potential risk of developing a given disease, disorder, or condition, and/or to assess the prognosis of the disease, disorder, or condition. The methods disclosed herein are also optionally used for characterizing a specific form of cancer. Since cancers are often heterogeneous in both composition and staging, the data generated using the methods disclosed herein may allow for the characterization of specific sub-types of cancer to thereby assist with diagnosis and treatment selection. This information may also provide a subject or healthcare practitioner with clues regarding the prognosis of a specific type of cancer, and enable a subject and/or healthcare practitioner to adapt treatment options in accordance with the progress of the disease. Some cancers become more aggressive and genetically unstable as they progress. Other tumors remain benign, inactive or dormant.

Samples

A sample can be any biological sample isolated from a subject. Samples can include body tissues, whole blood, platelets, serum, plasma, stool, red blood cells, white blood cells or leucocytes, endothelial cells, tissue biopsies (e.g., biopsies from known or suspected solid tumors), cerebrospinal fluid, synovial fluid, lymphatic fluid, ascites fluid, interstitial or extracellular fluid (e.g., fluid from intercellular spaces), gingival fluid, crevicular fluid, bone marrow, pleural effusions, cerebrospinal fluid, saliva, mucous, sputum, semen, sweat, urine. Samples are preferably body fluids, particularly blood and fractions thereof, and urine. Such samples include nucleic acids shed from tumors. The nucleic acids can include DNA and RNA and can be in double and single-stranded forms. A sample can be in the form originally isolated from a subject or can have been subjected to further processing to remove or add components, such as cells, enrich for one component relative to another, or convert one form of nucleic acid to another, such as RNA to DNA or single-stranded nucleic acids to double-stranded. Thus, for example, a body fluid sample for analysis is plasma or serum containing cell-free nucleic acids, e.g., cell-free DNA (cfDNA).

In some embodiments, the sample volume of body fluid taken from a subject depends on the desired read depth for sequenced regions. Exemplary volumes are about 0.4-40 ml, about 5-20 ml, about 10-20 ml. For example, the volume can be about 0.5 ml, about 1 ml, about 5 ml, about 10 ml, about 20 ml, about 30 ml, about 40 ml, or more milliliters. A volume of sampled plasma is typically between about 5 ml to about 20 ml.

The sample can comprise various amounts of nucleic acid. Typically, the amount of nucleic acid in a given sample is equated with multiple genome equivalents. For example, a sample of about 30 ng DNA can contain about 10,000 (10⁴) haploid human genome equivalents and, in the case of cfDNA, about 200 billion (2×10¹¹) individual polynucleotide molecules. Similarly, a sample of about 100 ng of DNA can contain about 30,000 haploid human genome equivalents and, in the case of cfDNA, about 600 billion individual molecules.

In some embodiments, a sample comprises nucleic acids from different sources, e.g., from cells and from cell-free sources (e.g., blood samples, etc.). Typically, a sample includes nucleic acids carrying mutations. For example, a sample optionally comprises DNA carrying germline mutations and/or somatic mutations. Typically, a sample comprises DNA carrying cancer-associated mutations (e.g., cancer-associated somatic mutations).

Exemplary amounts of cell-free nucleic acids in a sample before amplification typically range from about 1 femtogram (fg) to about 1 microgram (μg), e.g., about 1 picogram (pg) to about 200 nanogram (ng), about 1 ng to about 100 ng, about 10 ng to about 1000 ng. In some embodiments, a sample includes up to about 600 ng, up to about 500 ng, up to about 400 ng, up to about 300 ng, up to about 200 ng, up to about 100 ng, up to about 50 ng, or up to about 20 ng of cell-free nucleic acid molecules. Optionally, the amount is at least about 1 fg, at least about 10 fg, at least about 100 fg, at least about 1 pg, at least about 10 pg, at least about 100 pg, at least about 1 ng, at least about 10 ng, at least about 100 ng, at least about 150 ng, or at least about 200 ng of cell-free nucleic acid molecules. In certain embodiments, the amount is up to about 1 fg, about 10 fg, about 100 fg, about 1 pg, about 10 pg, about 100 pg, about 1 ng, about 10 ng, about 100 ng, about 150 ng, or about 200 ng of cell-free nucleic acid molecules. In some embodiments, methods include obtaining between about 1 fg to about 200 ng cell-free nucleic acid molecules from samples.

Cell-free nucleic acids typically have a size distribution of between about 100 nucleotides in length and about 500 nucleotides in length, with molecules of about 110 nucleotides in length to about 230 nucleotides in length representing about 90% of molecules in the sample, with a mode of about 168 nucleotides length and a second minor peak in a range between about 240 to about 440 nucleotides in length. In certain embodiments, cell-free nucleic acids are from about 160 to about 180 nucleotides in length, or from about 320 to about 360 nucleotides in length, or from about 440 to about 480 nucleotides in length.

In some embodiments, cell-free nucleic acids are isolated from bodily fluids through a partitioning step in which cell-free nucleic acids, as found in solution, are separated from intact cells and other non-soluble components of the bodily fluid. In some of these embodiments, partitioning includes techniques such as centrifugation or filtration. Alternatively, cells in bodily fluids are lysed, and cell-free and cellular nucleic acids processed together. Generally, after addition of buffers and wash steps, cell-free nucleic acids are precipitated with, for example, an alcohol. In certain embodiments, additional clean up steps are used, such as silica-based columns to remove contaminants or salts. Non-specific bulk carrier nucleic acids, for example, are optionally added throughout the reaction to optimize certain aspects of the exemplary procedure, such as yield. After such processing, samples typically include various forms of nucleic acids including double-stranded DNA, single-stranded DNA and/or single-stranded RNA. Optionally, single stranded DNA and/or single stranded RNA are converted to double stranded forms so that they are included in subsequent processing and analysis steps.

Nucleic Acid Tags

In certain embodiments, tags providing molecular identifiers or barcodes are incorporated into or otherwise joined to adapters by chemical synthesis, ligation, or overlap extension PCR, among other methods. In some embodiments, the assignment of unique or non-unique identifiers, or molecular barcodes in reactions follows methods and utilizes systems described in, for example, US patent applications 20010053519, 20030152490, 20110160078, and U.S. Pat. Nos. 6,582,908, 7,537,898, and 9,598,731, which are each incorporated by reference.

Tags are linked to sample nucleic acids randomly or non-randomly. In some embodiments, tags are introduced at an expected ratio of identifiers (e.g., a combination of unique and/or non-unique barcodes) to microwells. For example, the identifiers may be loaded so that more than about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers are loaded per genome sample. In some embodiments, the identifiers are loaded so that less than about 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers are loaded per genome sample. In certain embodiments, the average number of identifiers loaded per sample genome is less than, or greater than, about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers per genome sample. The identifiers are generally unique and/or non-unique.

One exemplary format uses from about 2 to about 1,000,000 different tags, or from about 5 to about 150 different tags, or from about 20 to about 50 different tags, ligated to both ends of a target nucleic acid molecule. For 20-50×20-50 tags, a total of 400-2500 tags are created. Such numbers of tags are typically sufficient for different molecules having the same start and stop points to have a high probability (e.g., at least 94%, 99.5%, 99.99%, 99.999%) of receiving different combinations of tags.

In some embodiments, identifiers are predetermined, random, or semi-random sequence oligonucleotides. In other embodiments, a plurality of barcodes may be used such that barcodes are not necessarily unique to one another in the plurality. In these embodiments, barcodes are generally attached (e.g., by ligation or PCR amplification) to individual molecules such that the combination of the barcode and the sequence it may be attached to creates a unique sequence that may be individually tracked. As described herein, detection of non-uniquely tagged barcodes in combination with sequence data of beginning (start) and end (stop) portions of sequence reads typically allows for the assignment of a unique identity to a particular molecule. The length, or number of base pairs, of an individual sequence read are also optionally used to assign a unique identity to a given molecule. As described herein, fragments from a single strand of nucleic acid having been assigned a unique identity, may thereby permit subsequent identification of fragments from the parent strand, and/or a complementary strand.

Nucleic Acid Amplification

Sample nucleic acids flanked by adapters are typically amplified by PCR and other amplification methods using nucleic acid primers binding to primer binding sites in adapters flanking a DNA molecule to be amplified. In some embodiments, amplification methods involve cycles of extension, denaturation and annealing resulting from thermocycling, or can be isothermal as, for example, in transcription mediated amplification. Other exemplary amplification methods that are optionally utilized, include the ligase chain reaction, strand displacement amplification, nucleic acid sequence-based amplification, and self-sustained sequence-based replication, among other approaches.

One or more rounds of amplification cycles are generally applied to introduce molecular tags and/or sample indexes/tags to a nucleic acid molecule using conventional nucleic acid amplification methods. The amplifications are typically conducted in one or more reaction mixtures. Molecular tags and sample indexes/tags are optionally introduced simultaneously, or in any sequential order. In some embodiments, molecular tags and sample indexes/tags are introduced prior to and/or after sequence capturing steps are performed. In some embodiments, only the molecular tags are introduced prior to probe capturing and the sample indexes/tags are introduced after sequence capturing steps are performed. In certain embodiments, both the molecular tags and the sample indexes/tags are introduced prior to performing probe-based capturing steps. In some embodiments, the sample indexes/tags are introduced after sequence capturing steps are performed. Typically, sequence capturing protocols involve introducing a single-stranded nucleic acid molecule complementary to a targeted nucleic acid sequence, e.g., a coding sequence of a genomic region and mutation of such region associated with a cancer type. Typically, the amplification reactions generate a plurality of non-uniquely or uniquely tagged nucleic acid amplicons with molecular tags and sample indexes/tags at size ranging from about 200 nucleotides (nt) to about 700 nt, from 250 nt to about 350 nt, or from about 320 nt to about 550 nt. In some embodiments, the amplicons have a size of about 300 nt. In some embodiments, the amplicons have a size of about 500 nt.

Nucleic Acid Enrichment

In some embodiments, sequences are enriched prior to sequencing the nucleic acids. Enrichment is optionally performed for specific target regions or nonspecifically (“target sequences”). In some embodiments, targeted regions of interest may be enriched with nucleic acid capture probes (“baits”) selected for one or more bait set panels using a differential tiling and capture scheme. A differential tiling and capture scheme generally uses bait sets of different relative concentrations to differentially tile (e.g., at different “resolutions”) across genomic sections associated with the baits, subject to a set of constraints (e.g., sequencer constraints such as sequencing load, utility of each bait, etc.), and capture the targeted nucleic acids at a desired level for downstream sequencing. These targeted genomic sections of interest optionally include natural or synthetic nucleotide sequences of the nucleic acid construct. In some embodiments, biotin-labeled beads with probes to one or more sections of interest can be used to capture target sequences, and optionally followed by amplification of those sections, to enrich for the regions of interest.

Sequence capture typically involves the use of oligonucleotide probes that hybridize to the target nucleic acid sequence. In certain embodiments, a probe set strategy involves tiling the probes across a section of interest. Such probes can be, for example, from about 60 to about 120 nucleotides in length. The set can have a depth of about 2×, 3×, 4×, 5×, 6×, 8×, 9×, 10×, 15×, 20×, 50× or more. The effectiveness of sequence capture generally depends, in part, on the length of the sequence in the target molecule that is complementary (or nearly complementary) to the sequence of the probe.

Nucleic Acid Sequencing

Sample nucleic acids, optionally flanked by adapters, with or without prior amplification are generally subject to sequencing. Sequencing methods or commercially available formats that are optionally utilized include, for example, Sanger sequencing, high-throughput sequencing, bisulfite sequencing, pyrosequencing, sequencing-by-synthesis, single-molecule sequencing, nanopore-based sequencing, semiconductor sequencing, sequencing-by-ligation, sequencing-by-hybridization, RNA-Seq (Illumina), Digital Gene Expression (Helicos), next generation sequencing (NGS), Single Molecule Sequencing by Synthesis (SMSS) (Helicos), massively-parallel sequencing, Clonal Single Molecule Array (Solexa), shotgun sequencing, Ion Torrent, Oxford Nanopore, Roche Genia, Maxim-Gilbert sequencing, primer walking, sequencing using PacBio, SOLiD, Ion Torrent, or nanopore platforms. Sequencing reactions can be performed in a variety of sample processing units, which may include multiple lanes, multiple channels, multiple wells, or other means of processing multiple sample sets substantially simultaneously. Sample processing units can also include multiple sample chambers to enable the processing of multiple runs simultaneously.

The sequencing reactions can be performed on one more nucleic acid fragment types or sections known to contain markers of cancer or of other diseases. The sequencing reactions can also be performed on any nucleic acid fragment present in the sample. The sequence reactions may provide for sequence coverage of the genome of at least about 5%, 10%, 15%, 20%, 25%, 30%, 40%, 50%, 60%, 70%, 80%, 90%, 95%, 99%, 99.9% or 100% of the genome. In other cases, sequence coverage of the genome may be less than about 5%, 10%, 15%, 20%, 25%, 30%, 40%, 50%, 60%, 70%, 80%, 90%, 95%, 99%, 99.9% or 100% of the genome.

Simultaneous sequencing reactions may be performed using multiplex sequencing techniques. In some embodiments, cell-free polynucleotides are sequenced with at least about 1000, 2000, 3000, 4000, 5000, 6000, 7000, 8000, 9000, 10000, 50000, or 100,000 sequencing reactions. In other embodiments, cell-free polynucleotides are sequenced with less than about 1000, 2000, 3000, 4000, 5000, 6000, 7000, 8000, 9000, 10000, 50000, or 100,000 sequencing reactions. Sequencing reactions are typically performed sequentially or simultaneously. Subsequent data analysis is generally performed on all or part of the sequencing reactions. In some embodiments, data analysis is performed on at least about 1000, 2000, 3000, 4000, 5000, 6000, 7000, 8000, 9000, 10000, 50000, or 100,000 sequencing reactions. In other embodiments, data analysis may be performed on less than about 1000, 2000, 3000, 4000, 5000, 6000, 7000, 8000, 9000, 10000, 50000, or 100,000 sequencing reactions. An exemplary read depth is from about 1000 to about 50000 reads per locus (base position).

In some embodiments, a nucleic acid population is prepared for sequencing by enzymatically forming blunt-ends on double-stranded nucleic acids with single-stranded overhangs at one or both ends. In these embodiments, the population is typically treated with an enzyme having a 5′-3′ DNA polymerase activity and a 3′-5′ exonuclease activity in the presence of the nucleotides (e.g., A, C, G and T or U). Exemplary enzymes or catalytic fragments thereof that are optionally used include Klenow large fragment and T4 polymerase. At 5′ overhangs, the enzyme typically extends the recessed 3′ end on the opposing strand until it is flush with the 5′ end to produce a blunt end. At 3′ overhangs, the enzyme generally digests from the 3′ end up to and sometimes beyond the 5′ end of the opposing strand. If this digestion proceeds beyond the 5′ end of the opposing strand, the gap can be filled in by an enzyme having the same polymerase activity that is used for 5′ overhangs. The formation of blunt-ends on double-stranded nucleic acids facilitates, for example, the attachment of adapters and subsequent amplification.

In some embodiments, nucleic acid populations are subject to additional processing, such as the conversion of single-stranded nucleic acids to double-stranded and/or conversion of RNA to DNA. These forms of nucleic acid are also optionally linked to adapters and amplified.

With or without prior amplification, nucleic acids subject to the process of forming blunt-ends described above, and optionally other nucleic acids in a sample, can be sequenced to produce sequenced nucleic acids. A sequenced nucleic acid can refer either to the sequence of a nucleic acid (i.e., sequence information) or a nucleic acid whose sequence has been determined. Sequencing can be performed so as to provide sequence data of individual nucleic acid molecules in a sample either directly or indirectly from a consensus sequence of amplification products of an individual nucleic acid molecule in the sample.

In some embodiments, double-stranded nucleic acids with single-stranded overhangs in a sample after blunt-end formation are linked at both ends to adapters including barcodes, and the sequencing determines nucleic acid sequences as well as in-line barcodes introduced by the adapters. The blunt-end DNA molecules are optionally ligated to a blunt end of an at least partially double-stranded adapter (e.g., a Y shaped or bell-shaped adapter). Alternatively, blunt ends of sample nucleic acids and adapters can be tailed with complementary nucleotides to facilitate ligation (e.g., sticky end ligation).

The nucleic acid sample is typically contacted with a sufficient number of adapters such that there is a low probability (e.g., <1 or 0.1%) that any two copies of the same nucleic acid receive the same combination of adapter barcodes from the adapters linked at both ends. The use of adapters in this manner permits identification of families of nucleic acid sequences with the same start and stop points on a reference nucleic acid and linked to the same combination of barcodes. Such a family represents sequences of amplification products of a nucleic acid in the sample before amplification. The sequences of family members can be compiled to derive consensus nucleotide(s) or a complete consensus sequence for a nucleic acid molecule in the original sample, as modified by blunt end formation and adapter attachment. In other words, the nucleotide occupying a specified position of a nucleic acid in the sample is determined to be the consensus of nucleotides occupying that corresponding position in family member sequences. Families can include sequences of one or both strands of a double-stranded nucleic acid. If members of a family include sequences of both strands from a double-stranded nucleic acid, sequences of one strand are converted to their complement for purposes of compiling all sequences to derive consensus nucleotide(s) or sequences. Some families include only a single member sequence. In this case, this sequence can be taken as the sequence of a nucleic acid in the sample before amplification. Alternatively, families with only a single member sequence can be eliminated from subsequent analysis.

Nucleotide variations in sequenced nucleic acids can be determined by comparing sequenced nucleic acids with a reference sequence. The reference sequence is often a known sequence, e.g., a known whole or partial genome sequence from a subject (e.g., a whole genome sequence of a human subject). The reference sequence can be, for example, hG19 or hG38. The sequenced nucleic acids can represent sequences determined directly for a nucleic acid in a sample, or a consensus of sequences of amplification products of such a nucleic acid, as described above. A comparison can be performed at one or more designated positions on a reference sequence. A subset of sequenced nucleic acids can be identified including a position corresponding with a designated position of the reference sequence when the respective sequences are maximally aligned. Within such a subset it can be determined which, if any, sequenced nucleic acids include a nucleotide variation at the designated position, the length of a given cfDNA fragment based upon where its endpoints (i.e., it 5′ and 3′ terminal nucleotides) map to the reference sequence, the offset of a midpoint of a given cfDNA fragment from a midpoint of a genomic region in the cfDNA fragment, and optionally which if any, include a reference nucleotide (i.e., same as in the reference sequence). If the number of sequenced nucleic acids in the subset including a nucleotide variant exceeding a selected threshold, then a variant nucleotide can be called at the designated position. The threshold can be a simple number, such as at least 1, 2, 3, 4, 5, 6, 7, 9, or 10 sequenced nucleic acids within the subset including the nucleotide variant or it can be a ratio, such as a least 0.5, 1, 2, 3, 4, 5, 10, 15, or 20 of sequenced nucleic acids within the subset that include the nucleotide variant, among other possibilities. The comparison can be repeated for any designated position of interest in the reference sequence. Sometimes a comparison can be performed for designated positions occupying at least about 20, 100, 200, or 300 contiguous positions on a reference sequence, e.g., about 20-500, or about 50-300 contiguous positions.

Additional details regarding nucleic acid sequencing, including the formats and applications described herein are also provided in, for example, Levy et al., Annual Review of Genomics and Human Genetics, 17: 95-115 (2016), Liu et al., J. of Biomedicine and Biotechnology, Volume 2012, Article ID 251364:1-11 (2012), Voelkerding et al., Clinical Chem., 55: 641-658 (2009), MacLean et al., Nature Rev. Microbiol., 7: 287-296 (2009), Astier et al., J Am Chem Soc., 128(5):1705-10 (2006), U.S. Pat. Nos. 6,210,891, 6,258,568, 6,833,246, 7,115,400, 6,969,488, 5,912,148, 6,130,073, 7,169,560, 7,282,337, 7,482,120, 7,501,245, 6,818,395, 6,911,345, 7,501,245, 7,329,492, 7,170,050, 7,302,146, 7,313,308, and 7,476,503, which are each incorporated by reference in their entirety.

Data Analysis

In some embodiments, raw sequencing data may comprise sets of sequence reads, which can be provided in various file formats, such as FASTQ, VCF, CRAM or BAM. Files with the raw sequencing data may include sequence data for one strand or both strands, such as in paired-end reads. In one example, the raw sequencing data is provided in a FASTQ file for both strands, i.e., sense and antisense strands generated from paired-end sequencing procedure. The files may include additional symbols providing information about the quality of reads and may also provide a quality score. The raw sequencing data of each polynucleotide molecule may be saved on a local drive, in cloud or a server.

In some cases, sequence reads generated from a sequencing reaction can be aligned or mapped to a reference sequence for carrying out bioinformatics analysis. The reference sequence is often a known sequence, e.g., a known whole or partial genome sequence from an object, whole genome sequence of a human subject. The reference sequence can be hG19. The sequenced nucleic acids can represent sequences determined directly for a nucleic acid in a sample, or a consensus of sequences of amplification products of such a nucleic acid, as described above. A comparison can be performed at one or more designated positions on a reference sequence.

Sequence reads may be aligned to a reference sequence using mapping tools, non-limiting examples of which may include Burrow's Wheeler Transform (BWA), Novoalign, and Bowtie. The mapping tools generate an alignment file describing alignment parameters used, position of the sequence reads (such as coordinates) on to the reference sequence and a quality score of mapping. The alignment parameters, such as number of differences allowed between the sequencing read and the reference sequence, number of gaps allowed and gap opening penalty, number of gap extensions, and the like, may be defined by a user. In one instance, BWA mapping tool with default alignment parameters is used to align the reads to a human reference genome, such as hg19. BWA tool provides an output file, a BAM file that includes alignment statistics. Alignment statistics may include coordinates of the reference sequence to which the processed reads align to. Alignment statistics may also provide a MapQ score to inform uniqueness of the reads when mapped to the reference sequence. The processed reads may then be sorted using the molecular barcodes and the coordinates on the reference sequence.

A subset of sequenced nucleic acids can be identified including a position corresponding with a designated position of the reference sequence when the respective sequences are maximally aligned. Within such a subset it can be determined which, if any, sequenced nucleic acids include a nucleotide variation at the designated position, and optionally which if any, include a reference nucleotide (i.e., same as in the reference sequence). The comparison can be repeated for any designated position of interest in the reference sequence. Sometimes a comparison can be performed for designated positions occupying at least 20, 100, 200, or 300 contiguous positions on a reference sequence, e.g., 20-500, or 50-300 contiguous positions.

A sample may be contacted with a sufficient number of different molecular barcodes that there is a low probability (e.g., <1 or 0.1%) that any two copies of the same nucleic acid receive the same combination of an adapter containing a molecular barcode from the adapters linked at one end or both ends. The use of adapters in this manner may permit grouping of sequence reads with the same start and stop points that are aligned (or mapped) to a reference sequence and linked to the same combination of molecular barcodes into families of reads generated from the same original molecule. Such a family may represent sequences of amplification products of a nucleic acid in the sample before amplification.

Sequences of family members can be compiled to derive consensus nucleotide(s) or a complete consensus sequence for a nucleic acid molecule in the original sample, as modified by blunt ending and adapter attachment. In other words, the nucleotide occupying a specified position of a nucleic acid in the sample may be determined to be the consensus of nucleotides occupying that corresponding position in family member sequences. A consensus nucleotide can be determined by methods such as voting or confidence score, to name two non-limiting, exemplary methods. Families can include sequences of one or both strands of a double-stranded nucleic acid. If members of a family include sequences of both strands from a double-stranded nucleic acid, sequences of one strand are converted to their complement for purposes of compiling all sequences to derive consensus nucleotide(s) or sequences. Some families may include only a single member sequence. In this case, this sequence can be taken as the sequence of a nucleic acid in the sample before amplification. Alternatively, families with only a single member sequence can be eliminated from subsequent analysis.

In some embodiments, the results of the systems and methods disclosed herein are used as an input to generate a report. The report may be in a paper format. For example, a report may provide an indication of the presence or absence of a therapeutic nucleic acid construct in a biological sample. In some embodiments, the report may include an indication of the level of the therapeutic nucleic acid construct in a biological sample.

The various steps of the methods disclosed herein, or the steps carried out by the systems disclosed herein, may be carried out at the same or different times, in the same or different geographical locations, e.g. countries, and/or by the same or different people.

Sequencing Panel

To improve the likelihood of detecting tumor indicating mutations, the region of DNA sequenced may comprise a panel of genes or genomic regions. Selection of a limited region for sequencing (e.g., a limited panel) can reduce the total sequencing needed (e.g., a total amount of nucleotides sequenced. A sequencing panel can target a plurality of different genes or regions to detect a single cancer, a set of cancers, or all cancers. Alternatively, DNA may be sequenced by whole genome sequencing (WGS) or other unbiased sequencing method without the use of a sequencing panel.

In some aspects, a panel that targets a plurality of different genes or genomic regions is selected such that a determined proportion of subjects having a cancer exhibits a genetic variant or tumor marker in one or more different genes in the panel. The panel may be selected to limit a region for sequencing to a fixed number of base pairs. The panel may be selected to sequence a desired amount of DNA. The panel may be further selected to achieve a desired sequence read depth. The panel may be selected to achieve a desired sequence read depth or sequence read coverage for an amount of sequenced base pairs. The panel may be selected to achieve a theoretical sensitivity, a theoretical specificity, and/or a theoretical accuracy for detecting one or more genetic variants in a sample.

Probes for detecting the panel of regions can include those for detecting genomic regions of interest (hotspot regions) as well as nucleosome-aware probes (e.g., KRAS codons 12 and 13) and may be designed to optimize capture based on analysis of cfDNA coverage and fragment size variation impacted by nucleosome binding patterns and GC sequence composition. Regions used herein can also include non-hotspot regions optimized based on nucleosome positions and GC models. The panel can comprise a plurality of subpanels, including subpanels for identifying tissue of origin (e.g., use of published literature to define 50-100 baits representing genes with most diverse transcription profile across tissues (not necessarily promoters)), whole genome scaffold (e.g., for identifying ultra-conservative genomic content and tiling sparsely across chromosomes with handful of probes for copy number base lining purposes), transcription start site (TSS)/CpG islands (e.g., for capturing differential methylated regions (e.g., Differentially Methylated Regions (DMRs)) in for example in promoters of tumor suppressor genes (e.g., SEPT9/VIM in colorectal cancer)). In some embodiments, markers for a tissue of origin are tissue-specific epigenetic markers.

Some examples of listings of genomic locations of interest may be found in Table 1 and Table 2. In some embodiments, genomic locations used in the methods of the present disclosure comprise at least a portion of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or 97 of the genes of Table 1. In some embodiments, genomic locations used in the methods of the present disclosure comprise at least 5, at least 10, at least 15, at least 20, at least 25, at least 30, at least 35, at least 40, at least 45, at least 50, at least 55, at least 60, at least 65, or 70 of the SNVs of Table 1. In some embodiments, genomic locations used in the methods of the present disclosure comprise at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the CNVs of Table 1. In some embodiments, genomic locations used in the methods of the present disclosure comprise at least 1, at least 2, at least 3, at least 4, at least 5, or 6 of the fusions of Table 1. In some embodiments, genomic locations used in the methods of the present disclosure comprise at least a portion of at least 1, at least 2, or 3 of the indels of Table 1. In some embodiments, genomic locations used in the methods of the present disclosure comprise at least a portion of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, or 115 of the genes of Table 2. In some embodiments, genomic locations used in the methods of the present disclosure comprise at least 5, at least 10, at least 15, at least 20, at least 25, at least 30, at least 35, at least 40, at least 45, at least 50, at least 55, at least 60, at least 65, at least 70, or 73 of the SNVs of Table 2. In some embodiments, genomic locations used in the methods of the present disclosure comprise at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the CNVs of Table 2. In some embodiments, genomic locations used in the methods of the present disclosure comprise at least 1, at least 2, at least 3, at least 4, at least 5, or 6 of the fusions of Table 2. In some embodiments, genomic locations used in the methods of the present disclosure comprise at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the indels of Table 2. Each of these genomic locations of interest may be identified as a backbone region or hot-spot region for a given bait set panel. An example of a listing of hot-spot genomic locations of interest may be found in Table 3. In some embodiments, genomic locations used in the methods of the present disclosure comprise at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 of the genes of Table 3. Each hot-spot genomic location is listed with several characteristics, including the associated gene, chromosome on which it resides, the start and stop position of the genome representing the gene's locus, the length of the gene's locus in base pairs, the exons covered by the gene, and the critical feature (e.g., type of mutation) that a given genomic location of interest may seek to capture.

TABLE 1

| Point Mutations (SNVs) | | | | | | Amplifications (CNVs) | | Fusions | Indels |
|---|---|---|---|---|---|---|---|---|---|
| AKT1 | ALK | APC | AR | ARAF | ARID1A | AR | BRAF | ALK | EGFR |
| ATM | BRAF | BRCA1 | BRCA2 | CCND1 | CCND2 | CCND1 | CCND2 | FGFR2 | (exons |
| CCNE1 | CDH1 | CDK4 | CDK6 | CDKN2A | CDKN2B | CCNE1 | CDK4 | FGFR3 | 19 & 20) |
| CTNNB1 | EGFR | ERBB2 | ESR1 | EZH2 | FBXW7 | CDK6 | EGFR | NTRK1 | ERBB2 |
| FGFR1 | FGFR2 | FGFR3 | GATA3 | GNA11 | GNAQ | ERBB2 | FGFR1 | RET | (exons |
| GNAS | HNF1A | HRAS | IDH1 | IDH2 | JAK2 | FGFR2 | KIT | ROS1 | 19 & 20) |
| JAK3 | KIT | KRAS | MAP2K1 | MAP2K2 | MET | KRAS | MET | | MET |
| MLH1 | MPL | MYC | NF1 | NFE2L2 | NOTCH1 | MYC | PDGFRA | | (exon 14 |
| NPM1 | NRAS | NTRK1 | PDGFRA | PIK3CA | PTEN | PIK3CA | RAF1 | | skipping) |
| PTPN11 | RAF1 | RB1 | RET | RHEB | RHOA | | | | |
| RIT1 | ROS1 | SMAD4 | SMO | SRC | STK11 | | | | |
| TERT | TP53 | TSC1 | VHL | | | | | | |

TABLE 2

| Point Mutations (SNVs) | | | | | | Amplifications (CNVs) | | Fusions | Indels |
|---|---|---|---|---|---|---|---|---|---|
| AKT1 | ALK | APC | AR | ARAF | ARID1A | AR | BRAF | ALK | EGFR |
| ATM | BRAF | BRCA1 | BRCA2 | CCND1 | CCND2 | CCND1 | CCND2 | FGFR2 | (exons |
| CCNE1 | CDH1 | CDK4 | CDK6 | CDKN2A | DDR2 | CCNE1 | CDK4 | FGFR3 | 19 & 20) |
| CTNNB1 | EGFR | ERBB2 | ESR1 | EZH2 | FBXW7 | CDK6 | EGFR | NTRK1 | ERBB2 |
| FGFR1 | FGFR2 | FGFR3 | GATA3 | GNA11 | GNAQ | ERBB2 | FGFR1 | RET | (exons |
| GNAS | HNF1A | HRAS | IDH1 | IDH2 | JAK2 | FGFR2 | KIT | ROS1 | 19 & 20) |
| JAK3 | KIT | KRAS | MAP2K1 | MAP2K2 | MET | KRAS | MET | | MET |
| MLH1 | MPL | MYC | NF1 | NFE2L2 | NOTCH1 | MYC | PDGFRA | | (exon 14 |
| NPM1 | NRAS | NTRK1 | PDGFRA | PIK3CA | PTEN | PIK3CA | RAF1 | | skipping) |
| PTPN11 | RAF1 | RB1 | RET | RHEB | RHOA | | | | ATM |
| RIT1 | ROS1 | SMAD4 | SMO | MAPK1 | STK11 | | | | |
| TERT | TP53 | TSC1 | VHL | MAPK3 | MTOR | | | | |
| NTRK3 | | | | | | | | | APC |
| | | | | | | | | | ARID1A |
| | | | | | | | | | BRCA1 |
| | | | | | | | | | BRCA2 |
| | | | | | | | | | CDH1 |
| | | | | | | | | | CDKN2A |
| | | | | | | | | | GATA3 |
| | | | | | | | | | KIT |
| | | | | | | | | | MLH1 |
| | | | | | | | | | MTOR |
| | | | | | | | | | NF1 |
| | | | | | | | | | PDGFRA |
| | | | | | | | | | PTEN |
| | | | | | | | | | RB1 |
| | | | | | | | | | SMAD4 |
| | | | | | | | | | STK11 |
| | | | | | | | | | TP53 |
| | | | | | | | | | TSC1 |
| | | | | | | | | | VHL |

TABLE 3

| | | Start | Stop | Length | Exons | Critical |
|---|---|---|---|---|---|---|
| Gene | Chromosome | Position | Position | (bp) | Covered | Feature |
| ALK | chr2 | 29446405 | 29446655 | 250 | intron 19 | Fusion |
| ALK | chr2 | 29446062 | 29446197 | 135 | intron 20 | Fusion |
| ALK | chr2 | 29446198 | 29446404 | 206 | 20 | Fusion |
| ALK | chr2 | 29447353 | 29447473 | 120 | intron 19 | Fusion |
| ALK | chr2 | 29447614 | 29448316 | 702 | intron 19 | Fusion |
| ALK | chr2 | 29448317 | 29448441 | 124 | 19 | Fusion |
| ALK | chr2 | 29449366 | 29449777 | 411 | intron 18 | Fusion |
| ALK | chr2 | 29449778 | 29449950 | 172 | 18 | Fusion |
| BRAF | chr7 | 140453064 | 140453203 | 139 | 15 | BRAF V600 |
| CTNNB1 | chr3 | 41266007 | 41266254 | 247 | 3 | S37 |
| EGFR | chr7 | 55240528 | 55240827 | 299 | 18 and 19 | G719 and deletions |
| EGFR | chr7 | 55241603 | 55241746 | 143 | 20 | Insertions/T790M |
| EGFR | chr7 | 55242404 | 55242523 | 119 | 21 | L858R |
| ERBB2 | chr17 | 37880952 | 37881174 | 222 | 20 | Insertions |
| ESR1 | chr6 | 152419857 | 152420111 | 254 | 10 | V534, P535, L536, |
| | | | | | | Y537, D538 |
| FGFR2 | chr10 | 123279482 | 123279693 | 211 | 6 | S252 |
| GATA3 | chr10 | 8111426 | 8111571 | 145 | 5 | SS/Indels |
| GATA3 | chr10 | 8115692 | 8116002 | 310 | 6 | SS/Indels |
| GNAS | chr20 | 57484395 | 57484488 | 93 | 8 | R844 |
| IDH1 | chr2 | 209113083 | 209113394 | 311 | 4 | R132 |
| IDH2 | chr15 | 90631809 | 90631989 | 180 | 4 | R140, R172 |
| KIT | chr4 | 55524171 | 55524258 | 87 | 1 | |
| KIT | chr4 | 55561667 | 55561957 | 290 | 2 | |
| KIT | chr4 | 55564439 | 55564741 | 302 | 3 | |
| KIT | chr4 | 55565785 | 55565942 | 157 | 4 | |
| KIT | chr4 | 55569879 | 55570068 | 189 | 5 | |
| KIT | chr4 | 55573253 | 55573463 | 210 | 6 | |
| KIT | chr4 | 55575579 | 55575719 | 140 | 7 | |
| KIT | chr4 | 55589739 | 55589874 | 135 | 8 | |
| KIT | chr4 | 55592012 | 55592226 | 214 | 9 | |
| KIT | chr4 | 55593373 | 55593718 | 345 | 10 and 11 | 557, 559, 560, 576 |
| KIT | chr4 | 55593978 | 55594297 | 319 | 12 and 13 | V654 |
| KIT | chr4 | 55595490 | 55595661 | 171 | 14 | T670, S709 |
| KIT | chr4 | 55597483 | 55597595 | 112 | 15 | D716 |
| KIT | chr4 | 55598026 | 55598174 | 148 | 16 | L783 |
| KIT | chr4 | 55599225 | 55599368 | 143 | 17 | C809, R815, D816, |
| | | | | | | L818, D820, S821F, |
| | | | | | | N822, Y823 |
| KIT | chr4 | 55602653 | 55602785 | 132 | 18 | A829P |
| KIT | chr4 | 55602876 | 55602996 | 120 | 19 | |
| KIT | chr4 | 55603330 | 55603456 | 126 | 20 | |
| KIT | chr4 | 55604584 | 55604733 | 149 | 21 | |
| KRAS | chr12 | 25378537 | 25378717 | 180 | 4 | A146 |
| KRAS | chr12 | 25380157 | 25380356 | 199 | 3 | Q61 |
| KRAS | chr12 | 25398197 | 25398328 | 131 | 2 | G12/G13 |
| MET | chr7 | 116411535 | 116412255 | 720 | 13, 14, | MET exon 14 SS |
| | | | | | intron 13, | |
| | | | | | intron 14 | |
| NRAS | chr1 | 115256410 | 115256609 | 199 | 3 | Q61 |
| NRAS | chr1 | 115258660 | 115258791 | 131 | 2 | G12/G13 |
| PIK3CA | chr3 | 178935987 | 178936132 | 145 | 10 | E545K |
| PIK3CA | chr3 | 178951871 | 178952162 | 291 | 21 | H1047R |
| PTEN | chr10 | 89692759 | 89693018 | 259 | 5 | R130 |
| SMAD4 | chr18 | 48604616 | 48604849 | 233 | 12 | D537 |
| TERT | chr5 | 1294841 | 1295512 | 671 | promoter | chr5: 1295228 |
| TP53 | chr17 | 7573916 | 7574043 | 127 | 11 | Q331, R337, R342 |
| TP53 | chr17 | 7577008 | 7577165 | 157 | 8 | R273 |
| TP53 | chr17 | 7577488 | 7577618 | 130 | 7 | R248 |
| TP53 | chr17 | 7578127 | 7578299 | 172 | 6 | R213/Y220 |
| TP53 | chr17 | 7578360 | 7578564 | 204 | 5 | R175/Deletions |
| TP53 | chr17 | 7579301 | 7579600 | 299 | 4 | |
| | | | | 12574 (total | | |
| | | | | target region) | | |
| | | | | 16330 (total | | |
| | | | | probe coverage) | | |

In some embodiments, the one or more regions in the panel comprise one or more loci from one or a plurality of genes for detecting residual cancer after surgery. This detection can be earlier than is possible for existing methods of cancer detection. In some embodiments, the one or more genomic locations in the panel comprise one or more loci from one or a plurality of genes for detecting cancer in a high-risk patient population. For example, smokers have much higher rates of lung cancer than the general population. Moreover, smokers can develop other lung conditions that make cancer detection more difficult, such as the development of irregular nodules in the lungs. In some embodiments, the methods described herein detect cancer in high risk patients earlier than is possible for existing methods of cancer detection.

A genomic location may be selected for inclusion in a sequencing panel based on a number of subjects with a cancer that have a tumor marker in that gene or region. A genomic location may be selected for inclusion in a sequencing panel based on prevalence of subjects with a cancer and a tumor marker present in that gene. Presence of a tumor marker in a region may be indicative of a subject having cancer.

In some instances, the panel may be selected using information from one or more databases. The information regarding a cancer may be derived from cancer tumor biopsies or cfDNA assays. A database may comprise information describing a population of sequenced tumor samples. A database may comprise information about mRNA expression in tumor samples. A databased may comprise information about regulatory elements or genomic regions in tumor samples. The information relating to the sequenced tumor samples may include the frequency various genetic variants and describe the genes or regions in which the genetic variants occur. The genetic variants may be tumor markers. A non-limiting example of such a database is COSMIC. COSMIC is a catalogue of somatic mutations found in various cancers. For a particular cancer, COSMIC ranks genes based on frequency of mutation. A gene may be selected for inclusion in a panel by having a high frequency of mutation within a given gene. For instance, COSMIC indicates that 33% of a population of sequenced breast cancer samples have a mutation in TP53 and 22% of a population of sampled breast cancers have a mutation in KRAS. Other ranked genes, including APC, have mutations found only in about 4% of a population of sequenced breast cancer samples. TP53 and KRAS may be included in a sequencing panel based on having relatively high frequency among sampled breast cancers (compared to APC, for example, which occurs at a frequency of about 4%). COSMIC is provided as a non-limiting example, however, any database or set of information may be used that associates a cancer with tumor marker located in a gene or genetic region. In another example, as provided by COSMIC, of 1156 biliary tract cancer samples, 380 samples (33%) carried mutations in TP53. Several other genes, such as APC, have mutations in 4-8% of all samples. Thus, TP53 may be selected for inclusion in the panel based on a relatively high frequency in a population of biliary tract cancer samples.

A gene or genomic section may be selected for a panel where the frequency of a tumor marker is significantly greater in sampled tumor tissue or circulating tumor DNA than found in a given background population. A combination of genomic locations may be selected for inclusion of a panel such that at least a majority of subjects having a cancer may have a tumor marker or genomic region present in at least one of the genomic location or genes in the panel. The combination of genomic location may be selected based on data indicating that, for a particular cancer or set of cancers, a majority of subjects have one or more tumor markers in one or more of the selected regions. For example, to detect cancer 1, a panel comprising regions A, B, C, and/or D may be selected based on data indicating that 90% of subjects with cancer 1 have a tumor marker in regions A, B, C, and/or D of the panel. Alternately, tumor markers may be shown to occur independently in two or more regions in subjects having a cancer such that, combined, a tumor marker in the two or more regions is present in a majority of a population of subjects having a cancer. For example, to detect cancer 2, a panel comprising regions X, Y, and Z may be selected based on data indicating that 90% of subjects have a tumor marker in one or more regions, and in 30% of such subjects a tumor marker is detected only in region X, while tumor markers are detected only in regions Y and/or Z for the remainder of the subjects for whom a tumor marker was detected. Tumor markers present in one or more genomic locations previously shown to be associated with one or more cancers may be indicative of or predictive of a subject having cancer if a tumor marker is detected in one or more of those regions 50% or more of the time. Computational approaches such as models employing conditional probabilities of detecting cancer given a cancer frequency for a set of tumor markers within one or more regions may be used to predict which regions, alone or in combination, may be predictive of cancer. Other approaches for panel selection involve the use of databases describing information from studies employing comprehensive genomic profiling of tumors with large panels and/or whole genome sequencing (WGS, RNA-seq, Chip-seq, bisulfate sequencing, ATAC-seq, and others). Information gleaned from literature may also describe pathways commonly affected and mutated in certain cancers. Panel selection may be further informed by the use of ontologies describing genetic information.

Genes included in the panel for sequencing can include the fully transcribed region, the promoter region, enhancer regions, regulatory elements, and/or downstream sequence. To further increase the likelihood of detecting tumor indicating mutations only exons may be included in the panel. The panel can comprise all exons of a selected gene, or only one or more of the exons of a selected gene. The panel may comprise of exons from each of a plurality of different genes. The panel may comprise at least one exon from each of the plurality of different genes.

In some aspects, a panel of exons from each of a plurality of different genes is selected such that a determined proportion of subjects having a cancer exhibit a genetic variant in at least one exon in the panel of exons.

At least one full exon from each different gene in a panel of genes may be sequenced. The sequenced panel may comprise exons from a plurality of genes. The panel may comprise exons from 2 to 100 different genes, from 2 to 70 genes, from 2 to 50 genes, from 2 to 30 genes, from 2 to 15 genes, or from 2 to 10 genes.

A selected panel may comprise a varying number of exons. The panel may comprise from 2 to 3000 exons. The panel may comprise from 2 to 1000 exons. The panel may comprise from 2 to 500 exons. The panel may comprise from 2 to 100 exons. The panel may comprise from 2 to 50 exons. The panel may comprise no more than 300 exons. The panel may comprise no more than 200 exons. The panel may comprise no more than 100 exons. The panel may comprise no more than 50 exons. The panel may comprise no more than 40 exons. The panel may comprise no more than 30 exons. The panel may comprise no more than 25 exons. The panel may comprise no more than 20 exons. The panel may comprise no more than 15 exons. The panel may comprise no more than 10 exons. The panel may comprise no more than 9 exons. The panel may comprise no more than 8 exons. The panel may comprise no more than 7 exons.

The panel may comprise one or more exons from a plurality of different genes. The panel may comprise one or more exons from each of a proportion of the plurality of different genes. The panel may comprise at least two exons from each of at least 25%, 50%, 75% or 90% of the different genes. The panel may comprise at least three exons from each of at least 25%, 50%, 75% or 90% of the different genes. The panel may comprise at least four exons from each of at least 25%, 50%, 75% or 90% of the different genes.

The sizes of the sequencing panel may vary. A sequencing panel may be made larger or smaller (in terms of nucleotide size) depending on several factors including, for example, the total amount of nucleotides sequenced or a number of unique molecules sequenced for a particular region in the panel. The sequencing panel can be sized 5 kb to 50 kb. The sequencing panel can be 10 kb to 30 kb in size. The sequencing panel can be 12 kb to 20 kb in size. The sequencing panel can be 12 kb to 60 kb in size. The sequencing panel can be at least 10 kb, 12 kb, 15 kb, 20 kb, 25 kb, 30 kb, 35 kb, 40 kb, 45 kb, 50 kb, 60 kb, 70 kb, 80 kb, 90 kb, 100 kb, 110 kb, 120 kb, 130 kb, 140 kb, or 150 kb in size. The sequencing panel may be less than 100 kb, 90 kb, 80 kb, 70 kb, 60 kb, or 50 kb in size.

The panel selected for sequencing can comprise at least 1, 5, 10, 15, 20, 25, 30, 40, 50, 60, 80, or 100 genomic locations (e.g., that each include genomic regions of interest). In some cases, the genomic locations in the panel are selected that the size of the locations are relatively small. In some cases, the regions in the panel have a size of about 10 kb or less, about 8 kb or less, about 6 kb or less, about 5 kb or less, about 4 kb or less, about 3 kb or less, about 2.5 kb or less, about 2 kb or less, about 1.5 kb or less, or about 1 kb or less or less. In some cases, the genomic locations in the panel have a size from about 0.5 kb to about 10 kb, from about 0.5 kb to about 6 kb, from about 1 kb to about 11 kb, from about 1 kb to about 15 kb, from about 1 kb to about 20 kb, from about 0.1 kb to about 10 kb, or from about 0.2 kb to about 1 kb. For example, the regions in the panel can have a size from about 0.1 kb to about 5 kb.

The panel selected herein can allow for deep sequencing that is sufficient to detect low-frequency genetic variants (e.g., in cell-free nucleic acid molecules obtained from a sample). An amount of genetic variants in a sample may be referred to in terms of the minor allele frequency for a given genetic variant. The minor allele frequency may refer to the frequency at which minor alleles (e.g., not the most common allele) occurs in a given population of nucleic acids, such as a sample. Genetic variants at a low minor allele frequency may have a relatively low frequency of presence in a sample. In some cases, the panel allows for detection of genetic variants at a minor allele frequency of at least 0.0001%, 0.001%, 0.005%, 0.01%, 0.05%, 0.1%, or 0.5%. The panel can allow for detection of genetic variants at a minor allele frequency of 0.001% or greater. The panel can allow for detection of genetic variants at a minor allele frequency of 0.01% or greater. The panel can allow for detection of genetic variant present in a sample at a frequency of as low as 0.0001%, 0.001%, 0.005%, 0.01%, 0.025%, 0.05%, 0.075%, 0.1%, 0.25%, 0.5%, 0.75%, or 1.0%. The panel can allow for detection of tumor markers present in a sample at a frequency of at least 0.0001%, 0.001%, 0.005%, 0.01%, 0.025%, 0.05%, 0.075%, 0.1%, 0.25%, 0.5%, 0.75%, or 1.0%. The panel can allow for detection of tumor markers at a frequency in a sample as low as 1.0%. The panel can allow for detection of tumor markers at a frequency in a sample as low as 0.75%. The panel can allow for detection of tumor markers at a frequency in a sample as low as 0.5%. The panel can allow for detection of tumor markers at a frequency in a sample as low as 0.25%. The panel can allow for detection of tumor markers at a frequency in a sample as low as 0.1%. The panel can allow for detection of tumor markers at a frequency in a sample as low as 0.075%. The panel can allow for detection of tumor markers at a frequency in a sample as low as 0.05%. The panel can allow for detection of tumor markers at a frequency in a sample as low as 0.025%. The panel can allow for detection of tumor markers at a frequency in a sample as low as 0.01%. The panel can allow for detection of tumor markers at a frequency in a sample as low as 0.005%. The panel can allow for detection of tumor markers at a frequency in a sample as low as 0.001%. The panel can allow for detection of tumor markers at a frequency in a sample as low as 0.0001%. The panel can allow for detection of tumor markers in sequenced cfDNA at a frequency in a sample as low as 1.0% to 0.0001%. The panel can allow for detection of tumor markers in sequenced cfDNA at a frequency in a sample as low as 0.01% to 0.0001%.

A genetic variant can be exhibited in a percentage of a population of subjects who have a disease (e.g., cancer). In some cases, at least 1%, 2%, 3%, 5%, 10%, 20%, 30%, 40%, 50%, 60%, 70%, 80%, 90%, 95%, or 99% of a population having the cancer exhibit one or more genetic variants in at least one of the regions in the panel. For example, at least 80% of a population having the cancer may exhibit one or more genetic variants in at least one of the genomic positions in the panel.

The panel can comprise one or more locations comprising genomic regions of interest from each of one or more genes. In some cases, the panel can comprise one or more locations comprising genomic regions of interest from each of at least 1, 2, 3, 4, 5, 6, 7, 8, 9, 10, 15, 20, 25, 30, 40, 50, or 80 genes. In some cases, the panel can comprise one or more locations comprising genomic regions of interest from each of at most 1, 2, 3, 4, 5, 6, 7, 8, 9, 10, 15, 20, 25, 30, 40, 50, or 80 genes. In some cases, the panel can comprise one or more locations comprising genomic regions of interest from each of from about 1 to about 80, from 1 to about 50, from about 3 to about 40, from 5 to about 30, from 10 to about 20 different genes.

The regions in the panel can be selected so that they comprise sequences differentially transcribed across one or more tissues. In some cases, the locations comprising genomic regions can comprise sequences transcribed in certain tissues at a higher level compared to other tissues. For example, the locations comprising genomic regions can comprise sequences transcribed in certain tissues but not in other tissues.

The genomic locations in the panel can comprise coding and/or non-coding sequences. For example, the genomic locations in the panel can comprise one or more sequences in exons, introns, promoters, 3′ untranslated regions, 5′ untranslated regions, regulatory elements, transcription start sites, and/or splice sites. In some cases, the regions in the panel can comprise other non-coding sequences, including pseudogenes, repeat sequences, transposons, viral elements, and telomeres. In some cases, the genomic locations in the panel can comprise sequences in non-coding RNA, e.g., ribosomal RNA, transfer RNA, Piwi-interacting RNA, and microRNA.

The genomic locations in the panel can be selected to detect (diagnose) a cancer with a desired level of sensitivity (e.g., through the detection of one or more genetic variants). For example, the regions in the panel can be selected to detect the cancer (e.g., through the detection of one or more genetic variants) with a sensitivity of at least 50%, 55%, 60%, 65%, 70%, 75%, 80%, 85%, 90%, 95%, 96%, 97%, 98%, 99%, 99.5%, or 99.9%. The genomic locations in the panel can be selected to detect the cancer with a sensitivity of 100%.

The genomic locations in the panel can be selected to detect (diagnose) a cancer with a desired level of specificity (e.g., through the detection of one or more genetic variants). For example, the genomic locations in the panel can be selected to detect cancer (e.g., through the detection of one or more genetic variants) with a specificity of at least 50%, 55%, 60%, 65%, 70%, 75%, 80%, 85%, 90%, 95%, 96%, 97%, 98%, 99%, 99.5%, or 99.9%. The genomic locations in the panel can be selected to detect the one or more genetic variant with a specificity of 100%.

The genomic locations in the panel can be selected to detect (diagnose) a cancer with a desired positive predictive value. Positive predictive value can be increased by increasing sensitivity (e.g., chance of an actual positive being detected) and/or specificity (e.g., chance of not mistaking an actual negative for a positive). As a non-limiting example, genomic locations in the panel can be selected to detect the one or more genetic variant with a positive predictive value of at least 50%, 55%, 60%, 65%, 70%, 75%, 80%, 85%, 90%, 95%, 96%, 97%, 98%, 99%, 99.5%, or 99.9%. The regions in the panel can be selected to detect the one or more genetic variant with a positive predictive value of 100%.

The genomic locations in the panel can be selected to detect (diagnose) a cancer with a desired accuracy. As used herein, the term “accuracy” may refer to the ability of a test to discriminate between a disease condition (e.g., cancer) and healthy condition. Accuracy may be can be quantified using measures such as sensitivity and specificity, predictive values, likelihood ratios, the area under the ROC curve, Youden's index and/or diagnostic odds ratio.

Accuracy may presented as a percentage, which refers to a ratio between the number of tests giving a correct result and the total number of tests performed. The regions in the panel can be selected to detect cancer with an accuracy of at least 50%, 55%, 60%, 65%, 70%, 75%, 80%, 85%, 90%, 95%, 96%, 97%, 98%, 99%, 99.5%, or 99.9%. The genomic locations in the panel can be selected to detect cancer with an accuracy of 100%.

A panel may be selected to be highly sensitive and detect low frequency genetic variants. For instance, a panel may be selected such that a genetic variant or tumor marker present in a sample at a frequency as low as 0.01%, 0.05%, or 0.001% may be detected at a sensitivity of at least 50%, 55%, 60%, 65%, 70%, 75%, 80%, 85%, 90%, 95%, 96%, 97%, 98%, 99%, 99.5%, or 99.9%. Genomic locations in a panel may be selected to detect a tumor marker present at a frequency of 1% or less in a sample with a sensitivity of 70% or greater. A panel may be selected to detect a tumor marker at a frequency in a sample as low as 0.1% with a sensitivity of at least 50%, 55%, 60%, 65%, 70%, 75%, 80%, 85%, 90%, 95%, 96%, 97%, 98%, 99%, 99.5%, or 99.9%. A panel may be selected to detect a tumor marker at a frequency in a sample as low as 0.01% with a sensitivity of at least 50%, 55%, 60%, 65%, 70%, 75%, 80%, 85%, 90%, 95%, 96%, 97%, 98%, 99%, 99.5%, or 99.9%. A panel may be selected to detect a tumor marker at a frequency in a sample as low as 0.001% with a sensitivity of at least 50%, 55%, 60%, 65%, 70%, 75%, 80%, 85%, 90%, 95%, 96%, 97%, 98%, 99%, 99.5%, or 99.9%.

A panel may be selected to be highly specific and detect low frequency genetic variants. For instance, a panel may be selected such that a genetic variant or tumor marker present in a sample at a frequency as low as 0.01%, 0.05%, or 0.001% may be detected at a specificity of at least 50%, 55%, 60%, 65%, 70%, 75%, 80%, 85%, 90%, 95%, 96%, 97%, 98%, 99%, 99.5%, or 99.9%. Genomic locations in a panel may be selected to detect a tumor marker present at a frequency of 1% or less in a sample with a specificity of 70% or greater. A panel may be selected to detect a tumor marker at a frequency in a sample as low as 0.1% with a specificity of at least 70%, 75%, 80%, 85%, 90%, 95%, 96%, 97%, 98%, 99%, 99.5%, or 99.9%. A panel may be selected to detect a tumor marker at a frequency in a sample as low as 0.01% with a specificity of at least 70%, 75%, 80%, 85%, 90%, 95%, 96%, 97%, 98%, 99%, 99.5%, or 99.9%. A panel may be selected to detect a tumor marker at a frequency in a sample as low as 0.001% with a specificity of at least 70%, 75%, 80%, 85%, 90%, 95%, 96%, 97%, 98%, 99%, 99.5%, or 99.9%.

A panel may be selected to be highly accurate and detect low frequency genetic variants. A panel may be selected such that a genetic variant or tumor marker present in a sample at a frequency as low as 0.01%, 0.05%, or 0.001% may be detected at an accuracy of at least 70%, 75%, 80%, 85%, 90%, 95%, 96%, 97%, 98%, 99%, 99.5%, or 99.9%. Genomic locations in a panel may be selected to detect a tumor marker present at a frequency of 1% or less in a sample with an accuracy of 70% or greater. A panel may be selected to detect a tumor marker at a frequency in a sample as low as 0.1% with an accuracy of at least 70%, 75%, 80%, 85%, 90%, 95%, 96%, 97%, 98%, 99%, 99.5%, or 99.9%. A panel may be selected to detect a tumor marker at a frequency in a sample as low as 0.01% with an accuracy of at least 70%, 75%, 80%, 85%, 90%, 95%, 96%, 97%, 98%, 99%, 99.5%, or 99.9%. A panel may be selected to detect a tumor marker at a frequency in a sample as low as 0.001% with an accuracy of at least 70%, 75%, 80%, 85%, 90%, 95%, 96%, 97%, 98%, 99%, 99.5%, or 99.9%.

A panel may be selected to be highly predictive and detect low frequency genetic variants. A panel may be selected such that a genetic variant or tumor marker present in a sample at a frequency as low as 0.01%, 0.05%, or 0.001% may have a positive predictive value of at least 70%, 75%, 80%, 85%, 90%, 95%, 96%, 97%, 98%, 99%, 99.5%, or 99.9%.

The concentration of probes or baits used in the panel may be increased (2 to 6 ng/μL) to capture more nucleic acid molecule within a sample. The concentration of probes or baits used in the panel may be at least 2 ng/μL, 3 ng/μL, 4 ng/μL, 5 ng/μL, 6 ng/μL, or greater. The concentration of probes may be about 2 ng/μL to about 3 ng/μL, about 2 ng/μL to about 4 ng/μL, about 2 ng/μL to about 5 ng/μL, about 2 ng/μL to about 6 ng/μL. The concentration of probes or baits used in the panel may be 2 ng/μL or more to 6 ng/μL or less. In some instances this may allow for more molecules within a biological to be analyzed thereby enabling lower frequency alleles to be detected.

Cancer and Other Diseases

In certain embodiments, the methods and aspects disclosed herein are used to diagnose a given disease, disorder or condition in patients. Typically, the disease under consideration is a type of cancer. Non-limiting examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic (CLL), chronic myeloid (CML), chronic myelomonocytic (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas. Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma.

Non-limiting examples of other genetic-based diseases, disorders, or conditions that are optionally evaluated using the methods and systems disclosed herein include achondroplasia, alpha-1 antitrypsin deficiency, antiphospholipid syndrome, autism, autosomal dominant polycystic kidney disease, Charcot-Marie-Tooth (CMT), cri du chat, Crohn's disease, cystic fibrosis, Dercum disease, down syndrome, Duane syndrome, Duchenne muscular dystrophy, Factor V Leiden thrombophilia, familial hypercholesterolemia, familial mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency (scid), sickle cell disease, spinal muscular atrophy, Tay-Sachs, thalassemia, trimethylaminuria, Turner syndrome, velocardiofacial syndrome, WAGR syndrome, Wilson disease, or the like.

Customized Therapies and Related Administration

In some embodiments, the methods disclosed herein relate to identifying and administering therapies to patients having a given disease, disorder or condition. Essentially any cancer therapy (e.g., surgical therapy, radiation therapy, chemotherapy, and/or the like) is included as part of these methods. Typically, therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In some embodiments, the immunotherapy or immunotherapeutic agents targets an immune checkpoint molecule. Certain tumors are able to evade the immune system by co-opting an immune checkpoint pathway. Thus, targeting immune checkpoints has emerged as an effective approach for countering a tumor's ability to evade the immune system and activating anti-tumor immunity against certain cancers. Pardoll, Nature Reviews Cancer, 2012, 12:252-264.

In certain embodiments, the immune checkpoint molecule is an inhibitory molecule that reduces a signal involved in the T cell response to antigen. For example, CTLA4 is expressed on T cells and plays a role in downregulating T cell activation by binding to CD80 (aka B7.1) or CD86 (aka B7.2) on antigen presenting cells. PD-1 is another inhibitory checkpoint molecule that is expressed on T cells. PD-1 limits the activity of T cells in peripheral tissues during an inflammatory response. In addition, the ligand for PD-1 (PD-L1 or PD-L2) is commonly upregulated on the surface of many different tumors, resulting in the downregulation of anti-tumor immune responses in the tumor microenvironment. In certain embodiments, the inhibitory immune checkpoint molecule is CTLA4 or PD-1. In other embodiments, the inhibitory immune checkpoint molecule is a ligand for PD-1, such as PD-L1 or PD-L2. In other embodiments, the inhibitory immune checkpoint molecule is a ligand for CTLA4, such as CD80 or CD86. In other embodiments, the inhibitory immune checkpoint molecule is lymphocyte activation gene 3 (LAG3), killer cell immunoglobulin like receptor (KIR), T cell membrane protein 3 (TIM3), galectin 9 (GAL9), or adenosine A2a receptor (A2aR).

Antagonists that target these immune checkpoint molecules can be used to enhance antigen-specific T cell responses against certain cancers. Accordingly, in certain embodiments, the immunotherapy or immunotherapeutic agent is an antagonist of an inhibitory immune checkpoint molecule. In certain embodiments, the inhibitory immune checkpoint molecule is PD-1. In certain embodiments, the inhibitory immune checkpoint molecule is PD-L1. In certain embodiments, the antagonist of the inhibitory immune checkpoint molecule is an antibody (e.g., a monoclonal antibody). In certain embodiments, the antibody or monoclonal antibody is an anti-CTLA4, anti-PD-1, anti-PD-L1, or anti-PD-L2 antibody. In certain embodiments, the antibody is a monoclonal anti-PD-1 antibody. In some embodiments, the antibody is a monoclonal anti-PD-L1 antibody. In certain embodiments, the monoclonal antibody is a combination of an anti-CTLA4 antibody and an anti-PD-1 antibody, an anti-CTLA4 antibody and an anti-PD-L1 antibody, or an anti-PD-L1 antibody and an anti-PD-1 antibody. In certain embodiments, the anti-PD-1 antibody is one or more of pembrolizumab (Keytruda®) or nivolumab (Opdivo®). In certain embodiments, the anti-CTLA4 antibody is ipilimumab (Yervoy®). In certain embodiments, the anti-PD-L1 antibody is one or more of atezolizumab (Tecentriq®), avelumab (Bavencio®), or durvalumab (Imfinzi®).

In certain embodiments, the immunotherapy or immunotherapeutic agent is an antagonist (e.g. antibody) against CD80, CD86, LAG3, KIR, TIM3, GAL9, or A2aR. In other embodiments, the antagonist is a soluble version of the inhibitory immune checkpoint molecule, such as a soluble fusion protein comprising the extracellular domain of the inhibitory immune checkpoint molecule and an Fc domain of an antibody. In certain embodiments, the soluble fusion protein comprises the extracellular domain of CTLA4, PD-1, PD-L1, or PD-L2. In some embodiments, the soluble fusion protein comprises the extracellular domain of CD80, CD86, LAG3, KIR, TIM3, GAL9, or A2aR. In one embodiment, the soluble fusion protein comprises the extracellular domain of PD-L2 or LAG3.

In certain embodiments, the immune checkpoint molecule is a co-stimulatory molecule that amplifies a signal involved in a T cell response to an antigen. For example, CD28 is a co-stimulatory receptor expressed on T cells. When a T cell binds to antigen through its T cell receptor, CD28 binds to CD80 (aka B7.1) or CD86 (aka B7.2) on antigen-presenting cells to amplify T cell receptor signaling and promote T cell activation. Because CD28 binds to the same ligands (CD80 and CD86) as CTLA4, CTLA4 is able to counteract or regulate the co-stimulatory signaling mediated by CD28. In certain embodiments, the immune checkpoint molecule is a co-stimulatory molecule selected from CD28, inducible T cell co-stimulator (ICOS), CD137, OX40, or CD27. In other embodiments, the immune checkpoint molecule is a ligand of a co-stimulatory molecule, including, for example, CD80, CD86, B7RP1, B7-H3, B7-H4, CD137L, OX40L, or CD70.

Agonists that target these co-stimulatory checkpoint molecules can be used to enhance antigen-specific T cell responses against certain cancers. Accordingly, in certain embodiments, the immunotherapy or immunotherapeutic agent is an agonist of a co-stimulatory checkpoint molecule. In certain embodiments, the agonist of the co-stimulatory checkpoint molecule is an agonist antibody and preferably is a monoclonal antibody. In certain embodiments, the agonist antibody or monoclonal antibody is an anti-CD28 antibody. In other embodiments, the agonist antibody or monoclonal antibody is an anti-ICOS, anti-CD137, anti-OX40, or anti-CD27 antibody. In other embodiments, the agonist antibody or monoclonal antibody is an anti-CD80, anti-CD86, anti-B7RP1, anti-B7-H3, anti-B7-H4, anti-CD137L, anti-OX40L, or anti-CD70 antibody.

Therapeutic options for treating specific genetic-based diseases, disorders, or conditions, other than cancer, are generally well-known to those of ordinary skill in the art and will be apparent given the particular disease, disorder, or condition under consideration.

In certain embodiments, the customized therapies described herein are typically administered parenterally (e.g., intravenously or subcutaneously). Pharmaceutical compositions containing the immunotherapeutic agent are typically administered intravenously. Certain therapeutic agents are administered orally. However, customized therapies (e.g., immunotherapeutic agents, etc.) may also be administered by any method known in the art, including, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

Systems and Computer Readable Media

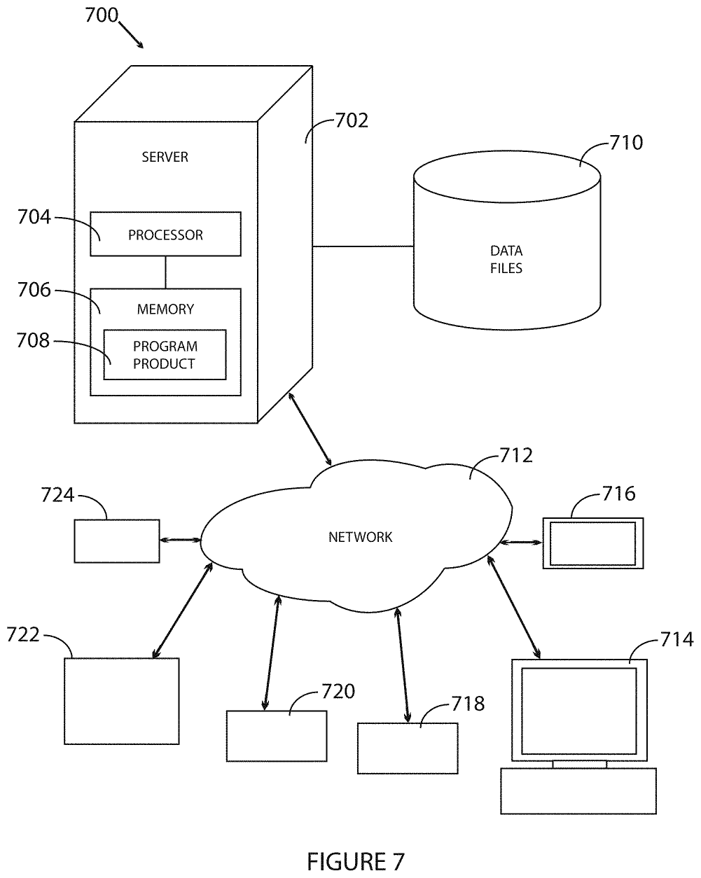

The present disclosure also provides various systems and computer program products or machine readable media. In some embodiments, for example, the methods described herein are optionally performed or facilitated at least in part using systems, distributed computing hardware and applications (e.g., cloud computing services), electronic communication networks, communication interfaces, computer program products, machine readable media, electronic storage media, software (e.g., machine-executable code or logic instructions) and/or the like. To illustrate, FIG. 7 provides a schematic diagram of an exemplary system suitable for use with implementing at least aspects of the methods disclosed in this application. As shown, system 700 includes at least one controller or computer, e.g., server 702 (e.g., a search engine server), which includes processor 704 and memory, storage device, or memory component 706, and one or more other communication devices 714 and 716 (e.g., client-side computer terminals, telephones, tablets, laptops, other mobile devices, etc.) positioned remote from and in communication with the remote server 702, through electronic communication network 712, such as the internet or other internetwork. Communication devices 714 and 716 typically include an electronic display (e.g., an internet enabled computer or the like) in communication with, e.g., server 702 computer over network 712 in which the electronic display comprises a user interface (e.g., a graphical user interface (GUI), a web-based user interface, and/or the like) for displaying results upon implementing the methods described herein. In certain embodiments, communication networks also encompass the physical transfer of data from one location to another, for example, using a hard drive, thumb drive, or other data storage mechanism. System 700 also includes program product 708 stored on a computer or machine readable medium, such as, for example, one or more of various types of memory, such as memory 706 of server 702, that is readable by the server 702, to facilitate, for example, a guided search application or other executable by one or more other communication devices, such as 714 (schematically shown as a desktop or personal computer) and 716 (schematically shown as a tablet computer). In some embodiments, system 700 optionally also includes at least one database server, such as, for example, server 710 associated with an online website having data stored thereon (e.g., classifier scores, control sample or comparator result data, indexed customized therapies, etc.) searchable either directly or through search engine server 702. System 700 optionally also includes one or more other servers positioned remotely from server 702, each of which are optionally associated with one or more database servers 710 located remotely or located local to each of the other servers. The other servers can beneficially provide service to geographically remote users and enhance geographically distributed operations.

As understood by those of ordinary skill in the art, memory 706 of the server 702 optionally includes volatile and/or nonvolatile memory including, for example, RAM, ROM, and magnetic or optical disks, among others. It is also understood by those of ordinary skill in the art that although illustrated as a single server, the illustrated configuration of server 702 is given only by way of example and that other types of servers or computers configured according to various other methodologies or architectures can also be used. Server 702 shown schematically in FIG. 7, represents a server or server cluster or server farm and is not limited to any individual physical server. The server site may be deployed as a server farm or server cluster managed by a server hosting provider. The number of servers and their architecture and configuration may be increased based on usage, demand and capacity requirements for the system 700. As also understood by those of ordinary skill in the art, other user communication devices 714 and 716 in these embodiments, for example, can be a laptop, desktop, tablet, personal digital assistant (PDA), cell phone, server, or other types of computers. As known and understood by those of ordinary skill in the art, network 712 can include an internet, intranet, a telecommunication network, an extranet, or world wide web of a plurality of computers/servers in communication with one or more other computers through a communication network, and/or portions of a local or other area network.

As further understood by those of ordinary skill in the art, exemplary program product or machine readable medium 708 is optionally in the form of microcode, programs, cloud computing format, routines, and/or symbolic languages that provide one or more sets of ordered operations that control the functioning of the hardware and direct its operation. Program product 708, according to an exemplary embodiment, also need not reside in its entirety in volatile memory, but can be selectively loaded, as necessary, according to various methodologies as known and understood by those of ordinary skill in the art.

As further understood by those of ordinary skill in the art, the term “computer-readable medium” or “machine-readable medium” refers to any medium that participates in providing instructions to a processor for execution. To illustrate, the term “computer-readable medium” or “machine-readable medium” encompasses distribution media, cloud computing formats, intermediate storage media, execution memory of a computer, and any other medium or device capable of storing program product 708 implementing the functionality or processes of various embodiments of the present disclosure, for example, for reading by a computer. A “computer-readable medium” or “machine-readable medium” may take many forms, including but not limited to, non-volatile media, volatile media, and transmission media. Non-volatile media includes, for example, optical or magnetic disks. Volatile media includes dynamic memory, such as the main memory of a given system. Transmission media includes coaxial cables, copper wire and fiber optics, including the wires that comprise a bus. Transmission media can also take the form of acoustic or light waves, such as those generated during radio wave and infrared data communications, among others. Exemplary forms of computer-readable media include a floppy disk, a flexible disk, hard disk, magnetic tape, a flash drive, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave, or any other medium from which a computer can read.

Program product 708 is optionally copied from the computer-readable medium to a hard disk or a similar intermediate storage medium. When program product 708, or portions thereof, are to be run, it is optionally loaded from their distribution medium, their intermediate storage medium, or the like into the execution memory of one or more computers, configuring the computer(s) to act in accordance with the functionality or method of various embodiments. All such operations are well known to those of ordinary skill in the art of, for example, computer systems.

To further illustrate, in certain embodiments, this application provides systems that include one or more processors, and one or more memory components in communication with the processor. The memory component typically includes one or more instructions that, when executed, cause the processor to provide information that causes sequence information, subclonality scores, classifier scores, test results, control or comparator results, customized therapies, and/or the like to be displayed (e.g., via communication devices 714, 716, or the like) and/or receive information from other system components and/or from a system user (e.g., via communication devices 714, 716, or the like).

In some embodiments, program product 708 includes non-transitory computer-executable instructions which, when executed by electronic processor 704 perform at least: (a) generating a subclonality score for each allele in a set of classification alleles from sequence information comprising sequence reads obtained from cell-free nucleic acid (cfNA) fragments from one or more reference samples, wherein each classification allele is of potential clinical significance and comprises a minor allele observed at a given locus in the reference samples, and b) comparing at least one selected cutoff threshold value to the subclonality scores, wherein classification alleles with subclonality scores above the selected cutoff threshold value indicate that those classification alleles are from reference cfNA fragments originating from non-target cells, which classification alleles are added to a non-target nucleic acid variant filter list, and/or wherein classification alleles with subclonality scores below the cutoff threshold value indicate that those classification alleles are from reference cfNA fragments originating from target cells, which classification alleles are added to a target nucleic acid variant filter list. Additional computer readable media embodiments are described herein.

System 700 also typically includes additional system components that are configured to perform various aspects of the methods described herein. In some of these embodiments, one or more of these additional system components are positioned remote from and in communication with the remote server 702 through electronic communication network 712, whereas in other embodiments, one or more of these additional system components are positioned local, and in communication with server 702 (i.e., in the absence of electronic communication network 712) or directly with, for example, desktop computer 714.

In some embodiments, for example, additional system components include sample preparation component 718 is operably connected (directly or indirectly (e.g., via electronic communication network 712)) to controller 702. Sample preparation component 718 is configured to prepare the nucleic acids in samples (e.g., prepare libraries of nucleic acids) to be amplified and/or sequenced by a nucleic acid amplification component (e.g., a thermal cycler, etc.) and/or a nucleic acid sequencer. In certain of these embodiments, sample preparation component 718 is configured to isolate nucleic acids from other components in a sample, to attach one or adapters comprising barcodes to nucleic acids as described herein, selectively enrich one or more regions from a genome or transcriptome prior to sequencing, and/or the like.

In certain embodiments, system 700 also includes nucleic acid amplification component 720 (e.g., a thermal cycler, etc.) operably connected (directly or indirectly (e.g., via electronic communication network 712)) to controller 702. Nucleic acid amplification component 720 is configured to amplify nucleic acids in samples from subjects. For example, nucleic acid amplification component 720 is optionally configured to amplify selectively enriched regions from a genome or transcriptome in the samples as described herein.

System 700 also typically includes at least one nucleic acid sequencer 722 operably connected (directly or indirectly (e.g., via electronic communication network 712)) to controller 702. Nucleic acid sequencer 722 is configured to provide the sequence information from nucleic acids (e.g., amplified nucleic acids) in samples from subjects. Essentially any type of nucleic acid sequencer can be adapted for use in these systems. For example, nucleic acid sequencer 722 is optionally configured to perform bisulfite sequencing, pyrosequencing, single-molecule sequencing, nanopore sequencing, semiconductor sequencing, sequencing-by-ligation, sequencing-by-hybridization, or other techniques on the nucleic acids to generate sequencing reads. Optionally, nucleic acid sequencer 722 is configured to group sequence reads into families of sequence reads, each family comprising sequence reads generated from a nucleic acid in a given sample. In some embodiments, nucleic acid sequencer 722 uses a clonal single molecule array derived from the sequencing library to generate the sequencing reads. In certain embodiments, nucleic acid sequencer 722 includes at least one chip having an array of microwells for sequencing a sequencing library to generate sequencing reads.

To facilitate complete or partial system automation, system 700 typically also includes material transfer component 724 operably connected (directly or indirectly (e.g., via electronic communication network 712)) to controller 702. Material transfer component 724 is configured to transfer one or more materials (e.g., nucleic acid samples, amplicons, reagents, and/or the like) to and/or from nucleic acid sequencer 722, sample preparation component 718, and nucleic acid amplification component 720.

Additional details relating to computer systems and networks, databases, and computer program products are also provided in, for example, Peterson, Computer Networks: A Systems Approach, Morgan Kaufmann, 5th Ed. (2011), Kurose, Computer Networking: A Top-Down Approach, Pearson, 7^{th} Ed. (2016), Elmasri, Fundamentals of Database Systems, Addison Wesley, 6th Ed. (2010), Coronel, Database Systems: Design, Implementation, & Management, Cengage Learning, 11th Ed. (2014), Tucker, Programming Languages, McGraw-Hill Science/Engineering/Math, 2nd Ed. (2006), and Rhoton, Cloud Computing Architected: Solution Design Handbook, Recursive Press (2011), which are each incorporated by reference in their entirety.

### EXAMPLES

### Example 1: Circulating Tumor Cell Free DNA

CtDNA (circulating tumor cell free DNA) in post-operative colorectal cancer (CRC) patients correlates with molecular residual disease and may be useful for prognostication and to guide adjuvant therapy decision making.

Post-operative ctDNA is strongly associated with disease recurrence in patients with metastatic CRC undergoing curative intent surgery (p=0.004), see Overman, et al. (2017). Circulating tumor DNA (ctDNA) can be found utilizing a high-sensitivity panel to detect minimal residual disease post liver hepatectomy and predict disease recurrence. JCO 35(suppl). Initial studies employed clinically impractical assays indexed to individual patient specific tumor tissue-derived mutations or were confounded by non-tumor-associated somatic alterations, including variants related to clonal hematopoiesis, see Tie J., et al. (2016). Sci Transl Med 8(346).

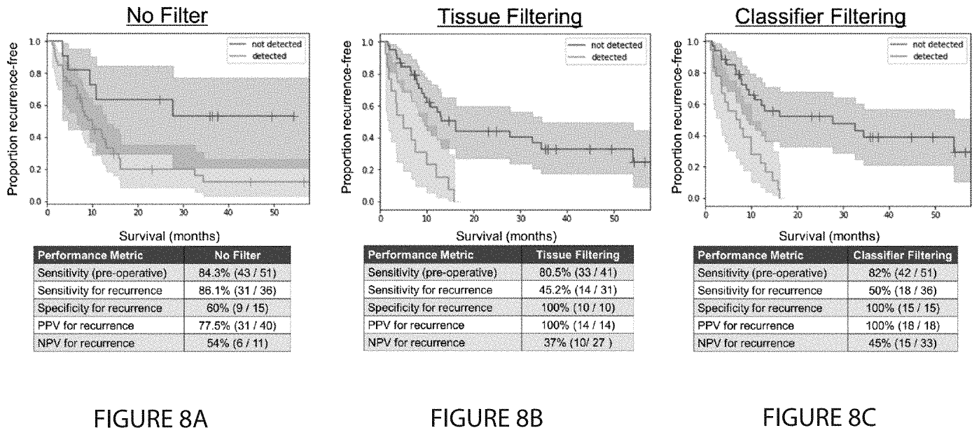

Data is provided showing that by using a highly sensitive CRC next-generation sequencing (NGS) panel, the detection of post-operative ctDNA does not require foreknowledge of known somatic alterations. A variant classifier was used to further differentiate between tumor-derived alterations from non-tumor derived alterations with the goal of increasing specificity of ctDNA detection in post-operative CRC patients (see Classifier filter in FIGS. 8A-C).

CRC patients planned for hepatic metastasectomy were prospectively enrolled in an IRB approved trial. Pre-operative and post-operative plasma was sequenced to high depth using a 38-gene NGS panel with 96% theoretical sensitivity for CRC. 51 metastatic colorectal cancer patients with both pre and post ctDNA results were recruited at a single institution (Table 4: Cohort Demographics). Tumor tissue was sequenced using this panel or local testing. ctDNA profiles from 17700 CRC pts (Guardant Health, Redwood City, Calif.) were used to train a variant classifier to exclude non-tumor derived alterations. The classifier was designed to identify cfDNA mutations that originate from the tumor.

TABLE 4

| | |
|---|---|
| Number of unique patients | 51 |
| Median age at diagnosis (range) | 55 years; range (33-76) |
| Gender | 60.8% Male |
| | 39.2% Female |
| Histological Grade | 98% Moderately Differentiated |
| | 2% Poorly Differentiated |
| Primary Site | 21.6% Right-sided |
| | 78.4% Left-sided |
| Presentation | 15.7% Metachronous |
| | 84.3% Synchronous |
| Neoadjuvant chemotherapy | 80.4% |
| Median number of resected tumors | 2 |
| Lymph node positive primary | 66.7% |
| KRAS mutation | 43% |
| Median time surgery to post- | 18 days (13-123 days) |
| operative sample | |
| Median follow-up | 42.7 months (range 4.4.-59.4 months) |
| Recurrence | 72.5% (37 patients) |
| Time to Recurrence (median) | 7.8 months (range 1.2-34.5 months) |

Recurrence prediction using post-operative somatic variant detection alone is fraught by a high clinical false positive rate. Many of the mutations from non-tumor origin occur at low allele frequencies However, a simple threshold on allele frequency would exclude many clinically relevant mutations Filtering using tumor tissue is effective but may be clinically impractical due to added complexity and cost. Filtering using a novel variant classifier, without foreknowledge of tumor genotype eliminated false positives while maintaining clinically acceptable sensitivity. A priori variant classification may enable clinically feasible ctDNA diagnostics for adjuvant decision making in early-stage disease.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, computer readable media, and/or component features, steps, elements, or other aspects thereof can be used in various combinations.

All patents, patent applications, websites, other publications or documents, accession numbers and the like cited herein are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number, if applicable. Likewise if different versions of a publication, website or the like are published at different times, the version most recently published at the effective filing date of the application is meant, unless otherwise indicated.

1. A method of detecting a nucleic acid molecule that originates from a target cell in a subject at least partially using a computer, the method comprising:
(a) receiving, by the computer, test sequence information comprising sequence reads obtained from cell-free nucleic acid (cfNA) fragments from a test sample obtained from the subject; and,
(b) identifying at least one allelic variant in the test sequence information;
(c) mapping the allelic variant to at least one classification allele on a target nucleic acid variant filter list;
(d) identifying a subclonality score of the classification allele; and,
(e) comparing the subclonality score to at least one selected cutoff threshold value, wherein when the subclonality score is below the selected cutoff threshold value it indicates that the classification allele is from a reference cfNA fragment that originates from the target cell, thereby detecting the nucleic acid molecule that originates from the target cell in the subject.

2-6. (canceled)

7. The method of claim 1, wherein identifying the set of classification alleles comprises determining a value of an MAF for each somatic nucleic acid variant at each locus in a set of target genomic loci of potential clinical significance from the sequence information obtained from the reference samples, wherein the set of target genomic loci is identical in each reference sample, and determining a value of a maxMAF for each of the reference samples, to generate allelic information.

8. The method of claim 7, wherein the MAF for each classification allele is less than about 2%.

9. The method of claim 8, wherein the MAF for each classification allele is less than about 1%.

10. (canceled)

11. The method of claim 1, comprising using clinical information indexed to the test sample to detect the nucleic acid molecule that originates from the target cell in the subject.

12. (canceled)

13. The method of claim 1, comprising determining subclonality scores using frequencies of each MAF/max-MAF value for each of the classification alleles.

14-17. (canceled)

18. The method of claim 1, comprising comparing the subclonality scores to multiple selected cutoff threshold values.

19. The method of claim 18, wherein the multiple selected cutoff threshold values comprise a first cutoff threshold value and a second cutoff threshold value, which first cutoff threshold value is greater than the second cutoff threshold value, wherein classification alleles with subclonality scores above the first cutoff threshold value indicate that those classification alleles are from reference cfNA fragments originating from non-target cells, which classification alleles are added to the non-target nucleic acid variant filter list, and/or wherein classification alleles with subclonality scores below the second cutoff threshold value indicate that those classification alleles are from reference cfNA fragments originating from target cells which classification alleles are added to the target nucleic acid variant filter list.

20. The method of claim 1, comprising classifying an allelic variant in the test sequence information that substantially matches at least one classification allele on a non-target nucleic acid variant filter list as originating from a target cell when the allelic variant comprises an MAF greater than about 1%.

21. The method of claim 1, comprising classifying an allelic variant in the test sequence information that substantially matches at least one classification allele on a non-target nucleic acid variant filter list as originating from a target cell when the allelic variant comprises a truncation, an indel, and/or a splice site variant.

22. The method of claim 1, comprising determining a frequency of each ratio value for a given classification allele in at least the portion of the reference samples.

23-24. (canceled)

25. A database comprising the target nucleic acid variant filter list and/or the non-target nucleic acid variant filter list of claim 1.

26. (canceled)

27. The method of claim 1, wherein the non-target cells comprise hematopoietic stem cells.

28. The method of claim 1, wherein the non-target cells comprise non-tumor cells.

29-31. (canceled)

32. The method of claim 1, wherein the target cells comprise tumor cells.

33-36. (canceled)

37. The method of claim 1, wherein the mammalian subject is a human subject.

38-40. (canceled)

41. The method of claim 1, further comprising amplifying segments of the cfNA fragments that comprise target genomic loci to generate amplified nucleic acids.

42. The method of claim 1, further comprising sequencing the cfNA fragments in the test sample to generate the test sequence information.

43. The method of claim 1, wherein the test sequence information is obtained from targeted segments of the cfNA fragments in the test sample, wherein the targeted segments are obtained by selectively enriching one or more regions from the cfNA fragments in the test sample prior to sequencing.

44. The method of claim 1, further comprising amplifying the obtained targeted segments prior to sequencing.

45-73. (canceled)
